(19) United States
(12) Patent Application Publication
(10) Pub. No.: US 2002/0115185 A1
(43) Pub. Date: Aug. 22, 2002

(54) Neuronal cell death associated molecules and uses therefor

(75) Inventors: Lillian W. Chiang, Edison, NJ (US); Andrew Wood, Newtown, PA (US); Lily Shiue, Santa Cruz, CA (US); Lorayne P. Jenkins, Monmouth Junction, NJ (US)

Correspondence Address: LAHIVE & COCKFIELD, 28 STATE STREET, BOSTON, MA, 02109, US

(73) Assignee: Millennium Pharmaceuticals, Inc., Cambridge, MA

(21) Appl. No.: 09/954,973

(22) Filed: Sep. 18, 2001

Related U.S. Application Data
(63) Continuation of application No. 09/393,174, filed on Sep. 9, 1999, now abandoned.
(60) Provisional application No. 60/099,616, filed on Sep. 9, 1998.

(51) Int. Cl. C12N 9/64; C07H 21/04; C12P 21/02; C12N 5/06

(52) U.S. Cl. 435/226; 435/69.1; 435/325; 435/320.1; 536/23.2

(57) ABSTRACT

The invention provides isolated nucleic acids molecules, designated CDAP nucleic acid molecules, which are differentially expressed in cells undergoing programmed cell death. The invention also provides antisense nucleic acid molecules, recombinant expression vectors containing CDAP nucleic acid molecules, host cells into which the expression vectors have been introduced, and nonhuman transgenic animals in which a CDAP gene has been introduced or disrupted. The invention still further provides isolated CDAP proteins, fusion proteins, antigenic peptides and anti-CDAP antibodies. Diagnostic methods utilizing compositions of the invention are also provided.

### RELATED APPLICATIONS

This application claims priority to U.S. provisional Application No. 60/099,616, filed on Sep.9, 1998, incorporated herein in its entirety by this reference.

### BACKGROUND OF THE INVENTION

During normal embryonic and adult development of multicellular organisms, cells that are not necessary or deleterious are eliminated by a process referred to as programmed cell death or apoptosis (Ellis R. E. et al. (1991) Annual Rev. Cell Biol. 7:663-698). Programmed cell death occurs both in vertebrate and invertebrate species and is characterized by unique morphological alterations, such as cytoplasmic contraction and chromatin condensation, as well as by specific DNA cleavage into oligonucleosomal fragments. Unlike necrosis, programmed cell death or apoptosis is an irreversible process which in most systems appears to depend on the expression of a specific set of novel “death genes”. Deregulation of this process contributes to the pathogenesis of several diseases including neurodegenerative disorders, cancer, immunodeficiency, and autoimmune diseases (Thompson C. B. et al. (1995) Science 267:1456).

In vertebrate species, neuronal programmed cell death mechanisms have been associated with a variety of developmental roles, including the removal of neuronal precursors which fail to establish appropriate synaptic connections (Oppenheim R. W. et al. (1991) Annual Rev. Neuroscience 14:453-501), the quantative matching of pre- and post-synaptic population sizes (Herrup K. et al. (1987) J. Neurosc. 7:829-836), and sculpting of neuronal circuits, both during development and in the adult (Bottjer S. W. et al. (1992) J. Neurobiol. 23:1172-1191).

Inappropriate apoptosis has been suggested to be involved in neuronal loss in various neurodegenerative diseases such as Alzheimer's disease (Loo et al. (1993) Proc. Natl. Acad. Sci. 90:7951-7955), Huntington's disease (Portera-Cailliau et al. (1995) J. Neurosc. 15:3775-3787), amyotrophic lateral sclerosis (Rabizadeh et al. (1995) Proc. Natl. Acad. Sci. 92:3024-3028), and spinal muscular atrophy (Roy et al. (1995) Cell 80:167-178).

### SUMMARY OF THE INVENTION

The present invention is based, at least in part, on the discovery of novel molecules which are differentially expressed during programmed cell death, e.g., neuronal programmed cell death, referred to herein as “cell death associated proteins” (“CDAP”) nucleic acid and protein molecules. The CDAP molecules of the present invention are useful as agents for diagnosing or prognosing subjects at risk for developing a disorder characterized by deregulated programmed cell death, e.g., a neurodegenerative disorder, as well as modulating agents in regulating a variety of cellular processes. Accordingly, in one aspect, this invention provides nucleic acid fragments suitable as primers or hybridization probes for the detection of CDAP-encoding nucleic acids as well as isolated nucleic acid molecules encoding CDAP proteins or biologically active portions thereof.

In one embodiment, a CDAP nucleic acid molecule of the invention is at least 44%, 50%, 55%, 60%, 65%, 70%, 75%, 80%, 85%, 89%, 90%, 95%, 98%, or more homologous to the nucleotide sequence (e.g., to the entire length of the nucleotide sequence) shown in SEQ ID NO: 1 or a complement thereof. In another embodiment, a CDAP nucleic acid molecule of the invention is at least 46%, 50%, 55%, 60%, 65%, 70%, 75%, 80%, 85%, 90%, 92%, 95%, 98% or more homologous to the nucleotide sequence (e.g., to the entire length of the nucleotide sequence) shown in SEQ ID NO:2 or a complement thereof. In another embodiment, a CDAP nucleic acid molecule of the invention is at least 50%, 55%, 60%, 65%, 70%, 75%, 80%, 82%, 85%, 90%, 95%, 98% or more homologous to the nucleotide sequence (e.g., to the entire length of the nucleotide sequence) shown in SEQ ID NO:3 or a complement thereof. In another embodiment, a CDAP nucleic acid molecule of the invention is at least 35%, 40%, 45%, 50%, 55%, 60%, 65%, 70%, 75%, 80%, 85%, 90%, 95%, 98% or more homologous to the nucleotide sequence (e.g., to the entire length of the nucleotide sequence) shown in SEQ ID NO:4 or a complement thereof. In another embodiment, a CDAP nucleic acid molecule of the invention is at least 60%, 65%, 70%, 75%, 80%, 85%, 90%, 94%, 95%, 98% or more homologous to the nucleotide sequence (e.g., to the entire length of the nucleotide sequence) shown in SEQ ID NO:5 or a complement thereof In yet another embodiment, a CDAP nucleic acid molecule of the invention is at least 60%, 65%, 70%; 75%, 80%, 85%, 90%, 95%, 98% or more homologous to the nucleotide sequence (e.g., to the entire length of the nucleotide sequence) shown in SEQ ID NO:7 or a complement thereof.

In a preferred embodiment, the isolated nucleic acid molecule includes the nucleotide sequence shown SEQ ID NO: 1 or a complement thereof. In another preferred embodiment, the nucleic acid molecule consists of the nucleotide sequence shown in SEQ ID NO: 1. In another preferred embodiment, the nucleic acid molecule includes a fragment of at least 154 nucleotides of the nucleotide sequence of SEQ ID NO: 1 or a complement thereof.

In a preferred embodiment, the isolated nucleic acid molecule includes the nucleotide sequence shown SEQ ID NO:2 or a complement thereof. In another preferred embodiment, the nucleic acid molecule consists of the nucleotide sequence shown in SEQ ID NO:2. In another preferred embodiment, the nucleic acid molecule includes a fragment of at least 168 nucleotides of the nucleotide sequence of SEQ ID NO:2 or a complement thereof.

In a preferred embodiment, the isolated nucleic acid molecule includes the nucleotide sequence shown SEQ ID NO:3 or a complement thereof. In another preferred embodiment, the nucleic acid molecule consists of the nucleotide sequence shown in SEQ ID NO:3. In another preferred embodiment, the nucleic.acid molecule includes a fragment of at least 110 nucleotides of the nucleotide sequence of SEQ ID NO:3 or a complement thereof.

In a preferred embodiment, the isolated nucleic acid molecule includes the nucleotide sequence shown SEQ ID NO:4 or a complement thereof. In another preferred embodiment, the nucleic acid molecule consists of the nucleotide sequence shown in SEQ ID NO:4. In another preferred embodiment, the nucleic acid molecule includes a fragment of at least 414 nucleotides of the nucleotide sequence of SEQ ID NO:4 or a complement thereof.

In a preferred embodiment, the isolated nucleic acid molecule includes the nucleotide sequence shown SEQ ID NO:5 or a complement thereof. In another preferred embodiment, the nucleic acid molecule consists of the nucleotide sequence shown in SEQ ID NO:5. In another preferred embodiment, the nucleic acid molecule includes a fragment of at least 420 nucleotides of the nucleotide sequence of SEQ ID NO:5 or a complement thereof.

In a preferred embodiment, the isolated nucleic acid molecule includes the nucleotide sequence shown SEQ ID NO:7 or a complement thereof. In another preferred embodiment, the nucleic acid molecule consists of the nucleotide sequence shown in SEQ ID NO:7.

Another embodiment of the invention features nucleic acid molecules, preferably CDAP nucleic acid molecules, which specifically detect CDAP nucleic acid molecules relative to nucleic acid molecules encoding non-CDAP proteins. For example, in one embodiment, such a nucleic acid molecule is at least 154, 200-250, 250-300, 300-350, 350-400,400-450,450-500 or more nucleotides in length and hybridizes under stringent conditions to a nucleic acid molecule comprising the nucleotide sequence shown in SEQ ID NO: 1 or a complement thereof. In preferred embodiments, the nucleic acid molecules are at least 15 (e.g., contiguous) nucleotides in length and hybridize under stringent conditions to nucleotides 1-5 89-102, or 346-403 of SEQ ID NO:1. In other preferred embodiments, the nucleic acid molecules comprise nucleotides 1-5, 89-102, or 346-403 of SEQ ID NO: 1.

In another particularly preferred embodiment, the nucleic acid molecule comprises a fragment of at least 168, 200-250, 250-300, 300-350, 350-400, 400-450,450-500 or more nucleotides of the nucleotide sequence of SEQ ID NO:2 or a complement thereof. In preferred embodiments, the nucleic acid molecules are at least 15 (e.g., contiguous) nucleotides in length and hybridize under stringent conditions to nucleotides 273-664 of SEQ ID NO:2. In other preferred embodiments, the nucleic acid molecules include, nucleotides 273-644 of SEQ ID NO:2.

In another particularly preferred embodiment, the nucleic acid molecule comprises a fragment of at least 110, 150-200, 200-250, 250-300, 300-350, 350-400, 400-450, 450-500 or more nucleotides of the nucleotide sequence of SEQ ID NO:3 or a complement thereof. In preferred embodiments, the nucleic acid molecules are at least 15 (e.g., contiguous) nucleotides in length and hybridize under stringent conditions to nucleotides 1-160, 317-351, or 453-585 of SEQ ID NO:3. In other preferred embodiments, the nucleic acid molecules include nucleotides 1-160, 317-351, or 453-585 of SEQ ID NO:3.

In another particularly preferred embodiment, the nucleic acid molecule comprises a fragment of at least 414, 450-500, 500-550, 550-600, 600-650 or more nucleotides of the nucleotide sequence of SEQ ID NO:4 or a complement thereof. In preferred embodiments, the nucleic acid molecules are at least 15 (e.g., contiguous) nucleotides in length and hybridize under stringent conditions to nucleotides 1-162, 288-490, or 589-644 of SEQ ID NO:4. In other preferred embodiments, the nucleic acid molecules include nucleotides 1-162, 288-490, or 589-644 of SEQ ID NO:4.

In another particularly preferred embodiment, the nucleic acid molecule comprises a fragment of at least 420, 450-500, 500-550, 550-600, 600-650 or more nucleotides of the nucleotide sequence of SEQ ID NO:5 or a complement thereof. In preferred embodiments, the nucleic acid molecules are at least 15 (e.g., contiguous) nucleotides in length and hybridize under stringent conditions to the nucleotide sequence of SEQ ID NO:5.

Another embodiment of the invention provides an isolated nucleic acid molecule which is antisense to a CDAP nucleic acid molecule, e.g., the coding strand of a CDAP nucleic acid molecule.

Another aspect of the invention provides a vector comprising a CDAP nucleic acid molecule. In certain embodiments, the vector is a recombinant expression vector. In another embodiment, the invention provides a host cell containing a vector of the invention.

In another aspect, the present invention provides a method for detecting the presence of a CDAP nucleic acid molecule, protein or polypeptide in a biological sample by contacting the biological sample with an agent capable of detecting a CDAP nucleic acid molecule, protein or polypeptide such that the presence of a CDAP nucleic acid molecule, protein or polypeptide is detected in the biological sample.

In another aspect, the present invention provides a method for detecting the presence of CDAP activity in a biological sample by contacting the biological sample with an agent capable of detecting an indicator of CDAP activity such that the presence of CDAP activity is detected in the biological sample.

In another aspect, the invention provides a method for modulating CDAP activity comprising contacting a cell capable of expressing CDAP with an agent that modulates CDAP activity such that CDAP activity in the cell is modulated. In one embodiment, the agent inhibits CDAP activity. In another embodiment, the agent stimulates CDAP activity. In one embodiment, the agent is an antibody that specifically binds to a CDAP protein. In another embodiment, the agent modulates expression of CDAP by modulating transcription of a CDAP gene or translation of a CDAP mRNA. In yet another embodiment, the agent is a nucleic acid molecule having a nucleotide sequence that is antisense to the coding strand of a CDAP mRNA or a CDAP gene.

In one embodiment, the methods of the present invention are used to treat a subject having a disorder characterized by aberrant CDAP protein or nucleic acid expression or activity by administering an agent which is a CDAP modulator to the subject. In one embodiment the CDAP modulator is a CDAP nucleic acid molecule. In another embodiment, the CDAP modulator is a CDAP protein. In yet another embodiment, the CDAP modulator is a peptide, peptidomimetic, or other small molecule. In a preferred embodiment, the disorder characterized by aberrant CDAP protein or nucleic acid expression is a disorder characterized by deregulated programmed cell death, e.g., a neurodegenerative disorder.

The present invention also provides a diagnostic assay for identifying the presence or absence of a genetic alteration characterized by at least one of (i) aberrant modification or mutation of a gene encoding a CDAP protein; (ii) mis-regulation of the gene; and (iii) aberrant post-translational modification of a CDAP protein, wherein a wild-type form of the gene encodes an protein with a CDAP activity.

In another embodiment, the invention features an isolated protein, preferably a CDAP protein, which is encoded by a nucleic acid molecule having a nucleotide sequence at least about 60%, 65%, 70%, 75%, 80%, 85%, 90%, 94%, 95%, 98% or more homologous to a nucleotide sequence of SEQ ID NO:5 or a complement thereof. This invention further features an isolated protein, preferably a CDAP protein, which is encoded by a nucleic acid molecule having a nucleotide sequence which hybridizes under stringent hybridization conditions to a nucleic acid molecule comprising the nucleotide sequence of SEQ ID NO:5 or a complement thereof.

The proteins of the present invention or biologically active portions thereof, can be operatively linked to a non-CDAP polypeptide (e.g., heterologous amino acid sequences) to form fusion proteins. The invention further features antibodies, such as monoclonal or polyclonal antibodies, that specifically bind proteins of the invention, preferably CDAP proteins. In addition, the CDAP proteins or biologically active portions thereof can be incorporated into pharmaceutical compositions, which optionally include pharmaceutically acceptable carriers.

In another embodiment, a CDAP nucleic acid molecule includes a nucleotide sequence encoding a protein having an amino acid sequence sufficiently homologous to the amino acid sequence of SEQ ID NO:6. In a preferred embodiment, a CDAP nucleic acid molecule includes a nucleotide sequence encoding a protein having an amino acid sequence at least 30%, 35%, 40%, 45%, 50%, 55%, 60%, 65%, 70%, 75%, 80%, 85%, 90%, 95% 98% or more homologous to the amino acid sequence of SEQ ID NO:6.

In another preferred embodiment, an isolated nucleic acid molecule encodes the amino acid sequence of human or rat CDAP-5. In yet another preferred embodiment, the nucleic acid molecule includes a nucleotide sequence encoding a protein having the amino acid sequence of SEQ ID NO: 6.

In other preferred embodiments, the nucleic acid molecule encodes a naturally occurring allelic variant of a polypeptide comprising the amino acid sequence of SEQ ID NO:6, wherein the nucleic acid molecule hybridizes to a nucleic acid molecule comprising SEQ ID NO:5 under stringent conditions.

Other features and advantages of the invention will be apparent from the following detailed description and claims.

### BRIEF DESCRIPTION OF THE DRAWINGS

FIG. 1 depicts the CDAP-1 nucleotide sequence (SEQ ID NO: 1).

FIG. 2 depicts the CDAP-2 nucleotide sequence (SEQ ID NO:2).

FIG. 3 depicts the CDAP-3 nucleotide sequence (SEQ ID NO:3).

FIG. 4 depicts the CDAP-4 nucleotide sequence (SEQ ID NO:4).

FIG. 5 depicts the CDAP-5 nucleotide (SEQ ID NO:5) and amino acid (SEQ ID NO:6) sequence.

FIG. 6 depicts the CDAP-6 nucleotide sequence (SEQ ID NO:7).

### DETAILED DESCRIPTION OF THE INVNETION

The present invention is based, at least in part, on the discovery of novel molecules, referred to herein as CDAP nucleic acid and protein molecules which are differentially expressed during programmed cell death, e.g., neuronal programmed cell death. The newly identified CDAP nucleic acid and protein molecules can be used to identify cells exhibiting or predisposed to a disorder characterized by deregulated programmed cell death, thereby diagnosing subjects having, or prone to developing such disorders.

As used herein, “programmed cell death” refers to a genetically regulated process involved in the normal development of multicellular organisms. This process occurs in cells destined for removal in a variety of normal situations, including larval development of the nematode c. elegans, insect metamorphosis, development in mammalian embryos including the nephrogenic zone in the developing kidney, and regression or atrophy (e.g., in the prostrate after castration). Programmed cell death can occur following the withdrawal of growth and trophic factors in many cells, nutritional deprivation, hormone treatment, ultraviolet irradiation, and exposure to toxic and infectious agents including reactive oxygen species and phosphatase inhibitors, e.g., okadaic acid, calcium ionphones, and a number of cancer chemotherapeutic agents. For a detailed description of programmed cell death see Trump B. F. et al. (1995) FASEB J. 9: 219-228 and Lee S. (1993) Curr. Opin. Cell Biol. 5:286-291, the contents of which are incorporated herein by reference. Thus, the CDAP proteins by being differentially expressed during programmed cell death, e.g., neuronal programmed cell death, can modulate a programmed cell death pathway activity and provide novel diagnostic targets and therapeutic agents for disorders characterized by deregulated programmed cell death, particularly in cells that express CDAP.

As used herein, a “disorder characterized by deregulated programmed cell death” refers to a disorder, disease or condition which is characterized by a deregulation, e.g., an upregulation or a downregulation, of programmed cell death. Programmed cell death deregulation can lead to deregulation of cellular proliferation and/or cell cycle progression. Examples of disorders characterized by deregulated programmed cell death include neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementias; ischemic injury, e.g., myocardial infarction, stroke, or reperfusion injury; or profilerative disorders, e.g., cancer.

As used herein, “differential expression”or differentially expressed” includes both quantative as well as qualitative differences in the temporal and/or cellular expression pattern of a gene, e.g., the CDAP-l, CDAP-2, CDAP-3, CDAP-4, CDAP-5, or CDAP-6 gene, among, for example, normal cells and cells undergoing programmed cell death. Genes which are differentially expressed can be used as part of a prognostic or diagnostic marker for the evaluation of subjects at risk for developing a disorder characterized by deregulated programmed cell death. Depending on the expression level of the gene, the progression state of the disorder can also be evaluated.

One embodiment of the invention features CDAP nucleic acid molecules, e.g., rat CDAP-1, CDAP-2, CDAP-3, CDAP4, CDAP-5, or CDAP-6, which were identified from rat cerebellar granule neurons undergoing programmed cell death, using rapid analysis of differential expression (RADETM). The CDAP-1 nucleic acid molecule is downregulated 2-fold at 3 hours after serum/potassium withdrawal. The CDAP-2 nucleic acid molecule is upregulated 4-fold at 3 hours after serum/potassium withdrawal. The CDAP-3 nucleic acid molecule is upregulated 3-fold at 3 hours after serum/potassium withdrawal. The CDAP-4 nucleic acid molecule is upregulated 6-fold at 3 hours after serum/potassium withdrawal. The CDAP-5 nucleic acid molecule is upregulated 5-fold at 3 hours after serum/potassium withdrawal. The CDAP-6 nucleic acid molecule is upregulated 4- to 16-fold at 3 hours after serum/potassium withdrawal.

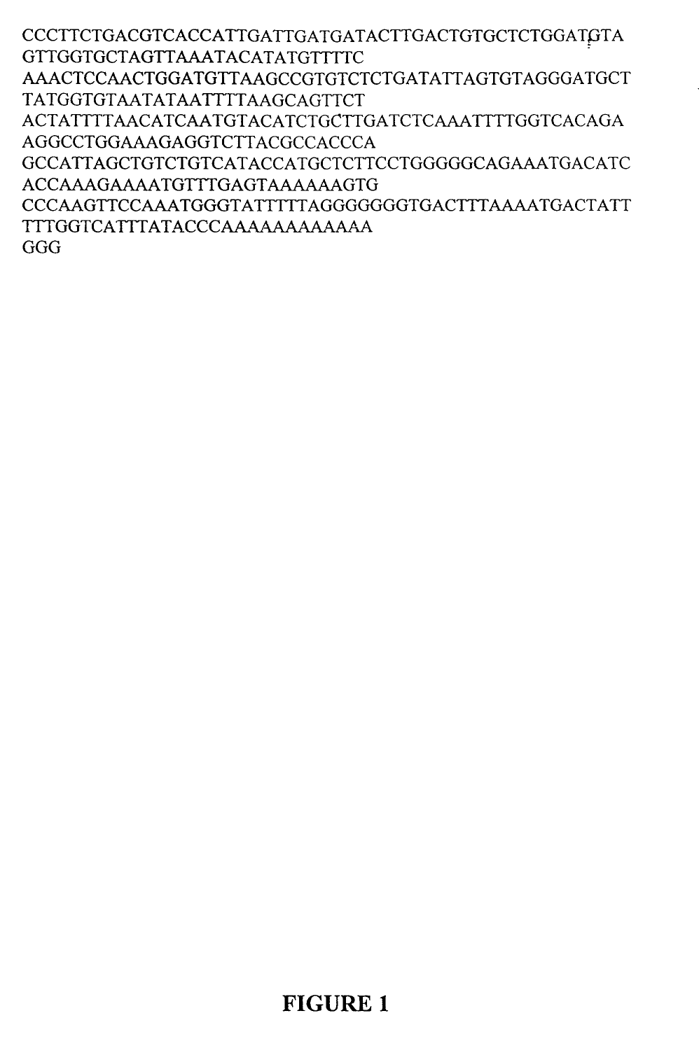

The nucleotide sequence of the isolated rat CDAP-1 cDNA is shown in FIG. 1 and in SEQ ID NO: 1. The CDAP-1 gene, which is approximately 403 nucleotides in length, is expressed in cerebellar granule neurons.

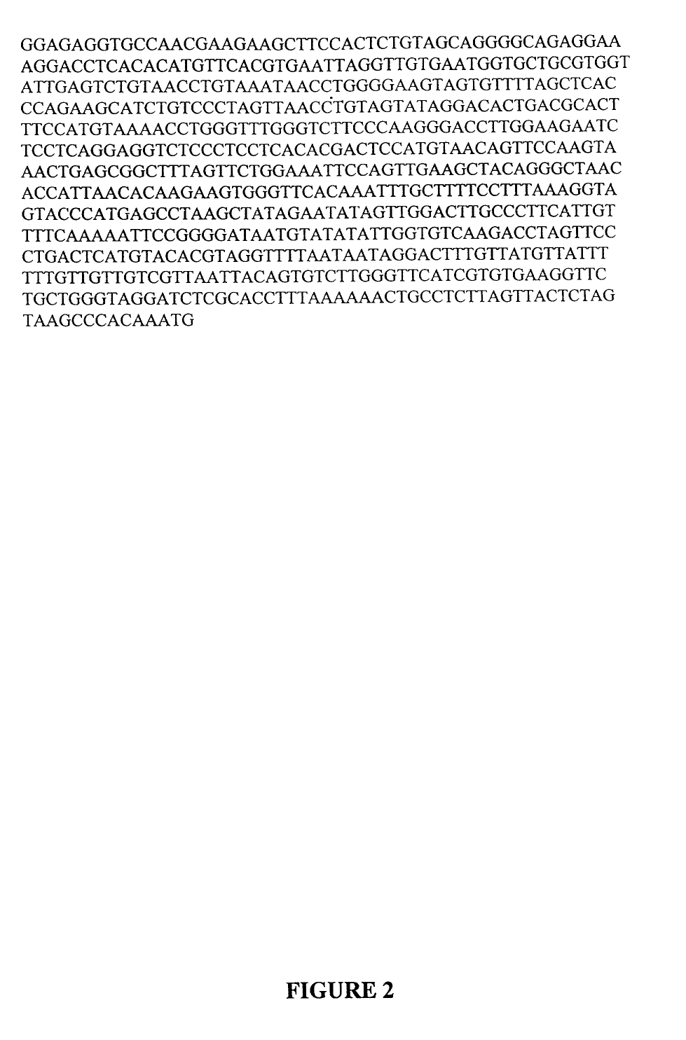

The nucleotide sequence of the isolated rat CDAP-2 cDNA is shown in FIG. 2 and in SEQ ID NO:2. The CDAP-2 gene, which is approximately 664 nucleotides in length, is expressed in the heart, brain, spleen, lung, testis, and smooth muscle.

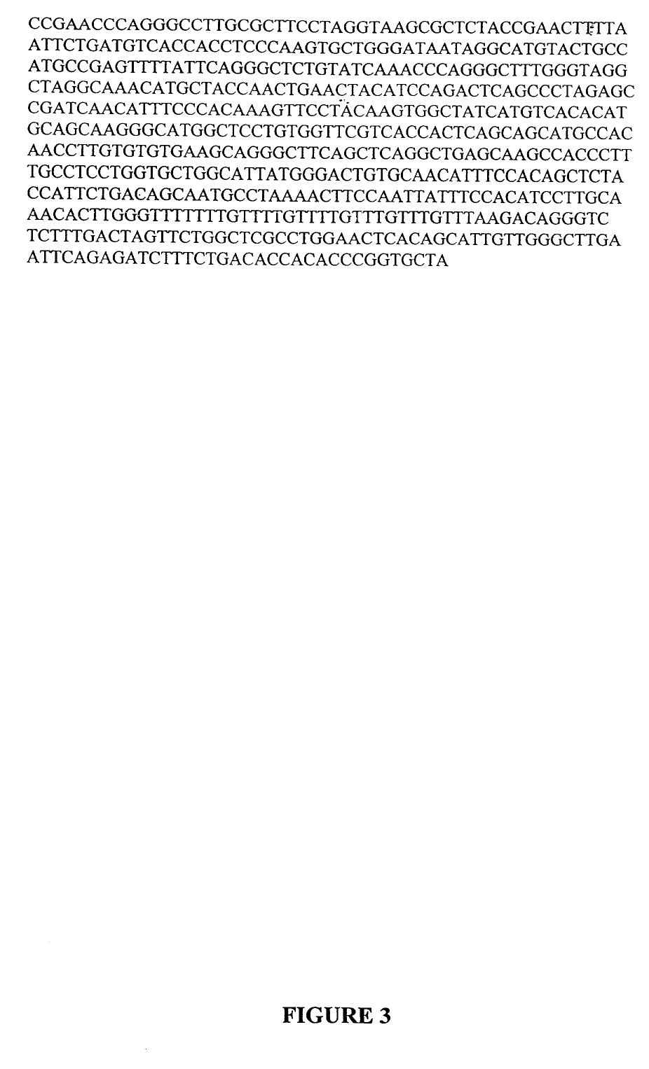

The nucleotide sequence of the isolated rat CDAP-3 cDNA is shown in FIG. 3 and in SEQ ID NO:3. The CDAP-3 gene, which is approximately 585 nucleotides in length, is expressed in the heart, spleen, lung, liver, skeletal muscle, kidney, testis, pancreas, and smooth muscle.

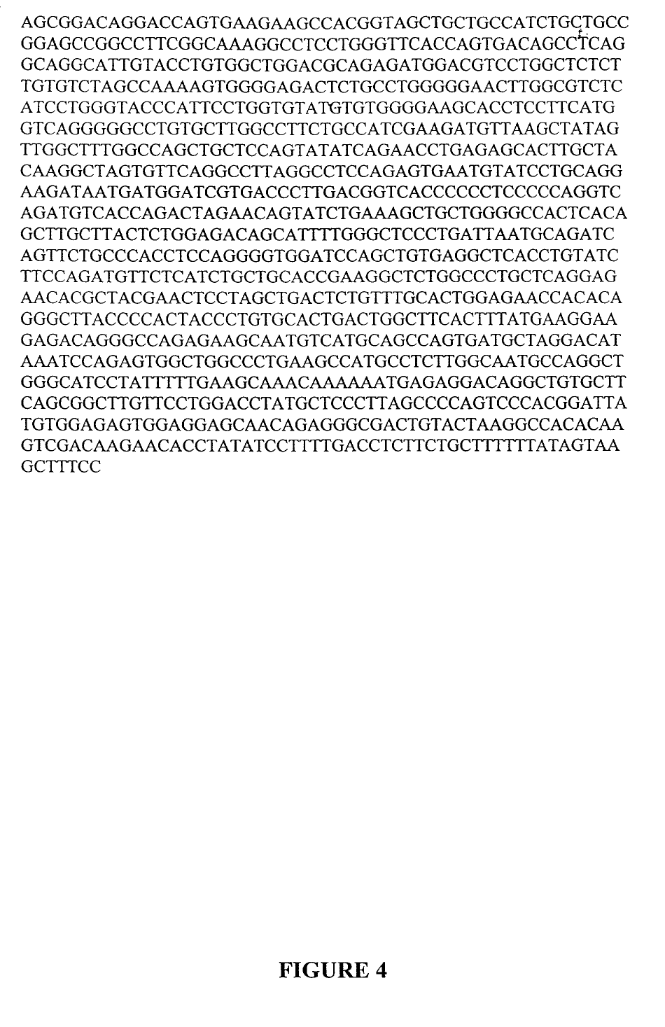

The nucleotide sequence of the isolated rat CDAP-4 cDNA is shown in FIG. 4 and in SEQ ID NO:4. The CDAP-4 gene, which is approximately 1057 nucleotides in length, is expressed in the brain (cerebellar granular cell layer), liver, kidney, and testis.

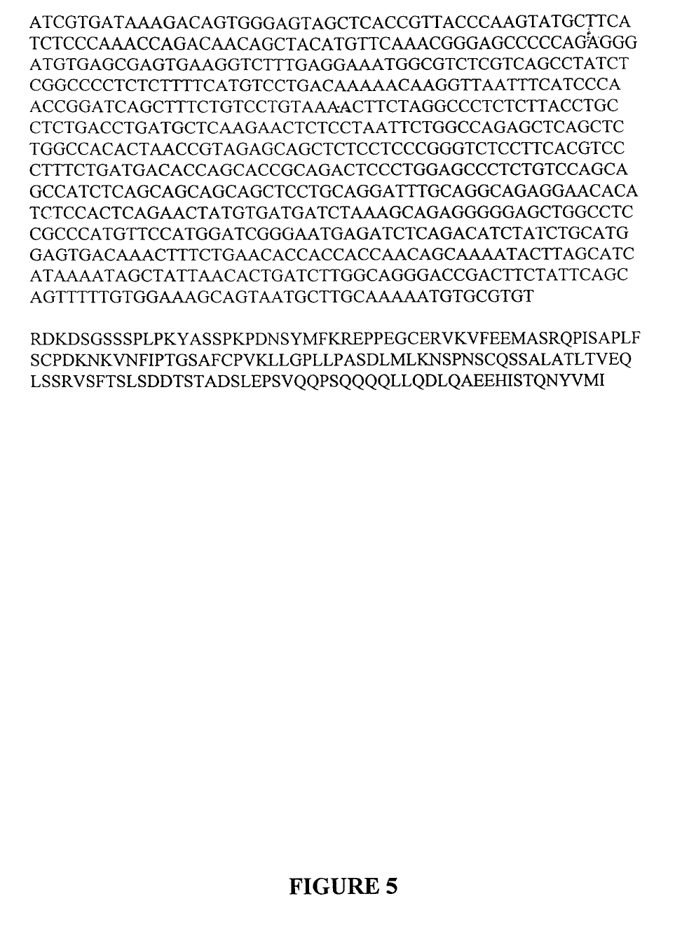

The nucleotide sequence of the isolated rat CDAP-5 cDNA and the predicted amino acid sequence of the rat CDAP-5 polypeptide are shown in FIG. 5 and in SEQ ID Nos.:5 and 6, respectively. The CDAP-5 gene, which is approximately 692 nucleotides in length, encodes a protein having a molecular weight of approximately 18.1 kD and which is approximately 158 amino acid residues in length. The CDAP-5 gene is expressed in the brain (cortex, hippocampus, cerebellar granular cell layer), eye, lung, brown fat, kidney, heart, spleen, liver, skeletal muscle, and kidney.

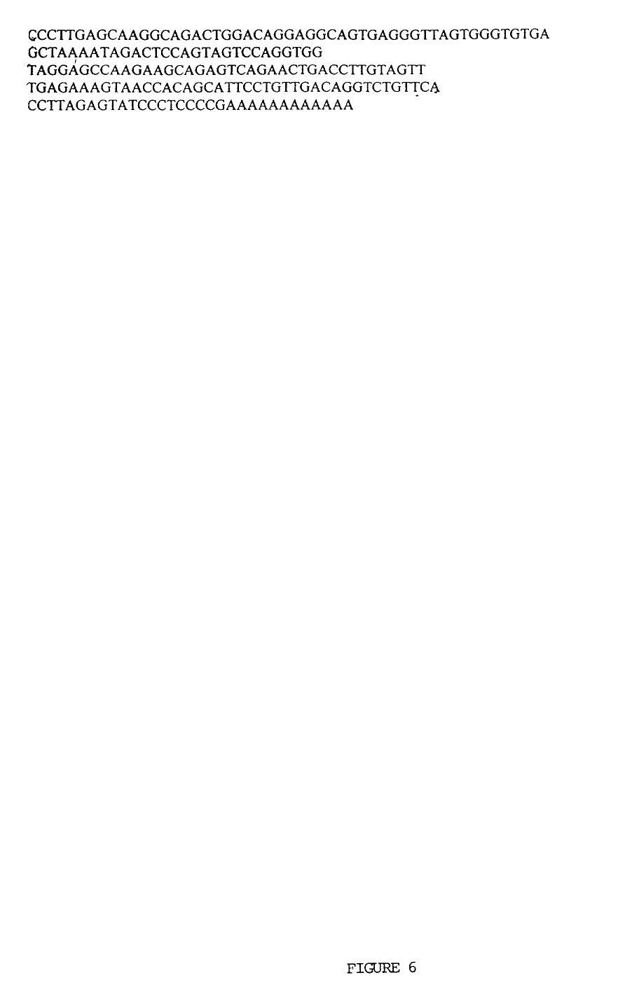

The nucleotide sequence of the isolated rat CDAP-6 cDNA is shown in FIG. 6 and in SEQ ID NO:7. The CDAP-6 gene, which is approximately 193 nucleotides in length, is expressed in cerebellar granule neurons.

Various aspects of the invention are described in further detail in the following subsections:

I. Differentially Expressed Nucleic Acid Molecules

One aspect of the invention pertains to isolated CDAP nucleic acid molecules or portions thereof, which are differentially expressed in cells, e.g., neuronal cells, undergoing programmed cell death. The invention further pertains to CDAP nucleic acid fragments sufficient for use as hybridization probes to identify CDAP-encoding nucleic acid molecules (e.g., CDAP mRNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CDAP nucleic acid molecule can contain less than about 5 kb, 4kb, 3kb, 2kb, 1 kb, 0.5 kb or 0.1 kb of nucleotide sequences which naturally flank the nucleic acid molecule in genomic DNA of the cell from which the nucleic acid is derived (e.g., a brain cell or other cell that expresses CDAP). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having the nucleotide sequence of SEQ ID NO: 1, 2, 3, 4, 5, or 7, or a portion thereof, can be isolated using standard molecular biology techniques and the sequence information provided herein. For example, a human CDAP cDNA can be isolated from a human brain library, using all or portion of SEQ ID NO: 1, 2, 3, 4, 5, or 7 as a hybridization probe and standard hybridization techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of SEQ ID NO: 1, 2, 3, 4, 5, or 7 can be isolated by the polymerase chain reaction using oligonucleotide primers designed based upon the sequence of SEQ ID NO: 1, 2, 3, 4, 5, or 7. For example, mRNA can be isolated from normal brain cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO: 1, 2, 3, 4, 5, or 7. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a CDAP nucleotide sequence can be prepared by standard synthetic techniques, e.g., using an automated DNA synthesizer.

In a preferred embodiment, an isolated nucleic acid molecule of the invention comprises the nucleotide sequence shown in SEQ ID NO: 1, 2, 3, 4, 5, or 7. The sequence of SEQ ID NO:1 corresponds to the rat CDAP-1 cDNA. The sequence of SEQ ID NO:2 corresponds to the rat CDAP-2 cDNA. The sequence of SEQ ID NO:3 corresponds, to the CDAP-3 cDNA. The sequence of SEQ ID NO:4 corresponds to the CDAP-4 cDNA. The sequence of SEQ ID NO:5 corresponds to the CDAP-5 cDNA. This cDNA comprises sequences encoding the rat CDAP-5 protein. The sequence of SEQ ID NO:7 corresponds to the CDAP-6 cDNA.

In another preferred embodiment, an isolated nucleic acid molecule of the invention comprises a nucleic acid molecule which is a complement of the nucleotide sequence shown in SEQ ID NO: 1, 2, 3, 4, 5, 7 or a portion of any of these nucleotide sequences. A nucleic acid molecule which is complementary to the nucleotide sequence shown in SEQ ID NO: 1, 2, 3, 4, 5, 7 is one which is sufficiently complementary to the nucleotide sequence shown in SEQ ID NO: 1, 2, 3, 4, 5, 7 such that it can hybridize to the nucleotide sequence shown in SEQ ID NO:1, 2, 3, 4, 5, 7 thereby forming a stable duplex.

In still another preferred embodiment, an isolated nucleic acid molecule of the present invention comprises a nucleotide sequence which is at least about 44%, 50%, 55%, 60%, 65%, 70%, 75%, 80%, 85%, 89%, 90%, 95%, 98% or more homologous to the entire length of the nucleotide sequence shown in SEQ ID NO: 1 or a portion thereof. In yet another preferred embodiment, an isolated nucleic acid molecule of the present invention comprises a nucleotide sequence which is at least about 46%, 50%, 55%, 60%, 65%, 70%, 75%, 80%, 85%, 90%, 92%, 95%, 98% or more homologous to the entire length of the nucleotide sequence shown in SEQ ID NO:2 or a portion thereof. In yet another preferred embodiment, an isolated nucleic acid molecule of the present invention comprises a nucleotide sequence which is at least about 50%, 55%, 60%, 65%, 70%, 75%, 80%, 82%, 85%, 90%, 95%, 98% or more homologous to the entire length of the nucleotide sequence shown in SEQ ID NO:3 or a portion thereof. In another preferred embodiment, an isolated nucleic acid molecule of the present invention comprises a nucleotide sequence which is at least about 35%, 40%, 45%, 50%, 55%, 60%, 65%, 70%, 75%, 80%, 85%, 90%, 95%, 98% or more homologous to the entire length of the nucleotide sequence shown in SEQ ID NO:4 or a portion thereof. In another preferred embodiment, an isolated nucleic acid molecule of the present invention comprises a nucleotide sequence which is at least about 60%, 65%, 70%, 75%, 80%, 85%, 90%, 94%, 95%, 98% or more homologous to the entire length of the nucleotide sequence shown in SEQ ID NO:5 or a portion thereof. In yet another preferred embodiment, an isolated nucleic acid molecule of the present invention comprises a nucleotide sequence which is at least about 60%, 65%, 70%, 75%, 80%, 85%, 90%, 95%, 98% or more homologous to the entire length of the nucleotide sequence shown in SEQ ID NO:7 or a portion thereof.

Moreover, the nucleic acid molecule of the invention can comprise only a portion of the nucleic acid sequence of SEQ ID NO: 1, 2, 3, 4, 5, or 7, for example a fragment which can be used as a probe or primer. The nucleotide sequence determined from the cloning of the CDAP gene allows for the generation of probes and primers designed for use in, identifying and/or cloning other CDAP family members, as well as CDAP homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO: 1, 2, 3, 4, 5, or 7, of an anti-sense sequence of SEQ ID NO: 1, 2, 3, 4, 5, or 7, or of a naturally occurring allelic variant or mutant of SEQ ID NO: 1, 2, 3, 4, 5, or 7. In an exemplary embodiment, a nucleic acid molecule of the present invention comprises a nucleotide sequence which is greater than 110, 154, 168, 200-250, 300-350, 350-400, 414, 420, 450-500, 500-550, 550-600, 600-650, 650-700, 700-750, 750-800, 800-850, 850-900, 950-1000, or more nucleotides in length and hybridizes under stringent hybridization conditions to a nucleic acid molecule of SEQ ID NO: 1, 2, 3, 4, 5, or 7.

Probes based on the CDAP nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic or prognostic test kit for identifying cells or tissue which misexpress or differentially express a CDAP protein, such as by measuring a level of a CDAP-encoding nucleic acid in a sample of cells from a subject e.g., detecting CDAP mRNA levels or determining whether a genomic CDAP gene has been mutated or deleted.

The invention further encompasses nucleic acid molecules that differ from the nucleotide sequence shown in SEQ ID NO: 1, 2, 3, 4, 5, or 7, due to degeneracy of the genetic code and thus encode the same CDAP proteins as those encoded by the nucleotide sequence shown in SEQ ID NO: 1, 2, 3, 4, 5, or 7. In another embodiment, an isolated nucleic acid molecule of the invention has a nucleotide sequence encoding a protein having an amino acid sequence shown in SEQ ID NO:6.

In addition to the CDAP nucleotide sequences shown in SEQ ID NO: 1, 2, 3, 4, 5, or 7, it will be appreciated by those skilled in the art that DNA sequence polymorphisms that lead to changes in the amino acid sequences of the CDAP proteins may exist within a population (e.g., the human population). Such genetic polymorphism in the CDAP genes may exist among individuals within a population due to natural allelic variation. Such natural allelic variations can typically result in 1-5% variance in the nucleotide sequence of a CDAP gene. Any and all such nucleotide variations and resulting amino acid polymorphisms in CDAP genes that are the result of natural allelic variation and that do not alter the functional activity of a CDAP protein are intended to be within the scope of the invention.

Moreover, nucleic acid molecules encoding other CDAP-1, CDAP-2, CDAP-3, CDAP-4, CDAP-5, CDAP-6, or CDAP-7 family members and, thus, which have a nucleotide sequence which differs from the CDAP sequences of SEQ ID NO:1, 2, 3, 4, 5, or 7 are intended to be within the scope of the invention. For example, another CDAP cDNA can be identified based on the nucleotide sequence of rat CDAP. Moreover, nucleic acid molecules encoding CDAP proteins from different species, and thus which have a nucleotide sequence which differs from the CDAP sequences of SEQ ID NO: 1, 2, 3, 4, 5, or 7 are intended to be within the scope of the invention. For example, a human CDAP cDNA can be identified based on the nucleotide sequence of a rat CDAP.

Nucleic acid molecules corresponding to natural allelic variants and homologues of the CDAP cDNAs of the invention can be isolated based on their homology to the CDAP nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:l, 2, 3, 4, 5, or 7. In other embodiments, the nucleic acid is at least 30, 50, 100, 150, 200, 250, 300, 350, 400, 450, 500, 550, 600, 650, 700, 750, 800, 850, 900, or 950 nucleotides in length. As used herein, the term “hybridizes under stringent conditions” is intended to describe conditions for hybridization and washing under which nucleotide sequences of a particular length and nucleic acid content will remain hybridized to each other. Stringent conditions are conditions which favor hybridization of complementary sequences of nucleic acid molecules. Homologous or related nucleic acid sequences will remain hybridized under stringent conditions. For example, stringent hybridization conditions are such that sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6X sodium chloride/sodium citrate (SSC) at about 45° C., followed by one or more washes in 0.2 X SSC, 0.1% SDS at 50° C., 55° C., 60° C., or 65° C. Preferably, an isolated nucleic acid molecule of the invention that hybridizes under stringent conditions to the sequence of SEQ ID NO: 1 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to the nucleic acid molecules encoding CDAP proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire CDAP coding strand, or to only a portion thereof. In one embodiment, an antisense nucleic acid molecule is antisense to a “coding region” of the coding strand of a nucleotide sequence encoding CDAP. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding CDAP. The term “noncoding region” refers to 5′ and 3′ sequences which flank the coding region that are not translated into amino acids (i.e., also referred to as 5′ and 3′ untranslated regions).

Given the CDAP nucleic acid moleculles disclosed herein (e.g., SEQ ID NO:1, 2, 3, 4, 5, or 7), antisense nucleic acids of the invention can be designed according to the rules of Watson and Crick base pairing. The antisense nucleic acid molecule can be complementary to the entire CDAP nucleic acid molecule, but more preferably is an oligonucleotide which is antisense to only a portion of the CDAP nucleic acid molecule. For example, the antisense oligonucleotide can be complementary to the region surrounding the translation start site of CDAP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a CDAP protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an cc-anomeric nucleic acid molecule. An (x-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave CDAP mRNA transcripts to thereby inhibit translation of CDAP MnRNA. A ribozyme having specificity for a CDAP-encoding nucleic acid can be designed based upon the nucleotide sequence of a CDAP cDNA disclosed herein (i.e., SEQ ID NO: 1, 2, 3, 4, 5, or 7). For example, a derivative of a Tetrahymena L-19 IVS RNA can be constructed in which the nucleotide sequence of the active site is complementary to the nucleotide sequence to be cleaved in a CDAP-encoding mRNA. See, e.g., Cech et al. U.S. Pat. No. 4,987,071; and Cech et al. U.S. Pat. No. 5,116,742. Alternatively, CDAP MRNA can be used to select a catalytic RNA having a specific ribonuclease activity from a pool of RNA molecules. See, e.g., Bartel, D. and Szostak, J. W. (1993) Science 261:1411-1418.

Alternatively, CDAP gene expression can be inhibited by targeting nucleotide sequences complementary to the regulatory region of the CDAP (e.g., the CDAP promoter and/or enhancers) to form triple helical structures that prevent transcription of the CDAP gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the CDAP nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of CDAP nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of CDAP nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of CDAP can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of CDAP nucleic acid molecules can be generated which may combine the advantageous properties of PNA and DNA. Such chimeras allow DNA recognition enzymes, (e.g., RNAse H and DNA, polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P.J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. W088/098 10) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

In addition to naturally-occurring allelic variants of the CDAP sequences that may exist in the population, the skilled artisan will further appreciate that changes can be introduced by mutation into the nucleotide sequences of SEQ ID NO: 1, 2, 3, 4, 5, or 7, thereby leading to changes in the amino acid sequence of the encoded CDAP proteins, without altering the functional ability of the CDAP proteins. For example, nucleotide substitutions leading to amino acid substitutions at “non-essential” amino acid residues can be made in the sequence of SEQ ID NO: 1, 2, 3, 4, 5, or 7. A “non-essential” amino acid residue is a residue that can be altered from the wild-type sequence of CDAP (e.g., the sequence of SEQ ID NO:6) without altering the biological activity, whereas an “essential” amino acid residue is required for biological activity. For example, amino acid residues that are conserved among the CDAP proteins of the present invention, are predicted to be particularly unamenable to alteration.

Accordingly, another aspect of the invention pertains to nucleic acid molecules encoding CDAP proteins that contain changes in amino acid residues that are not essential for activity. Such CDAP proteins differ in amino acid sequence from SEQ ID NO:6, yet retain biological activity. In one embodiment, the isolated nucleic acid molecule comprises a nucleotide sequence encoding a protein, wherein the protein comprises an amino acid sequence at least about 30%, 35%, 40%, 45%, 50%, 55%, 60%, 65%, 70%, 75%, 80%, 85%, 90%, 95% 98% or more homologous to SEQ ID NO:6.

An isolated nucleic acid molecule encoding a CDAP protein homologous to the protein of SEQ ID NO:6 can be created by introducing one or more nucleotide substitutions, additions or deletions into the nucleotide sequence of SEQ ID NO: 1, 2, 3, 4, 5, or 7, such that one or more amino acid substitutions, additions or deletions are introduced into the encoded protein. Mutations can be introduced into SEQ ID NO:1, 2, 3, 4, 5, or 7 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a CDAP protein is preferably replaced with another amino acid residue from the same side chain family. Alternatively, in another embodiment, mutations can be introduced randomly along all or part of a CDAP coding sequence, such as by saturation mutagenesis, and the resultant mutants can be screened for CDAP biological activity to identify mutants that retain activity. Following mutagenesis of SEQ ID NO:1, 2, 3, 4, 5, or 7, the encoded protein can be expressed recombinantly and the activity of the protein can be determined.

In a preferred embodiment, a mutant CDAP protein can be assayed for the ability to (1) interact with a non-CDAP protein molecule; (2) activate a CDAP-dependent cell activity; and (3) modulate programmed cell death in a cell, e.g., a cell which expresses CDAP.

II. Uses and Methods of the Invention

The compositions described herein, particularly the differentially expressed CDAP nucleic acid molecules, as well as the proteins, protein homologues, and antibodies can be used in one or more of the following methods: a) predictive medicine (e.g., diagnostic assays, prognostic assays, monitoring clinical trials, and pharmacogenetics); b) methods of treatment (e.g., therapeutic and prophylactic); c) detection assays; and d) screening assays. As described herein, CDAP nucleic acid molecules are differentially expressed in cells, e.g., neuronal cells, undergoing programmed cell death. Accordingly, the isolated nucleic acid molecules of the invention can be used to, for example, detect CDAP mRNA (e.g., in a biological sample) or a genetic alteration in a CDAP gene, to thereby diagnose subjects having, or prone to developing a disorder characterized by deregulated programmed cell death such as a neurodegenerative disorder, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementias; ischemic injury, e.g., myocardial infarction, stroke, or reperfusion injury; or profilerative disorders, e.g., cancer. The isolated nucleic acid molecules of the invention can further be used to modulate CDAP activity, or express CDAP protein (e.g., via a recombinant expression vector in a host cell in gene therapy applications), as described further below.

A. Predictive Medicine:

The present invention pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining CDAP nucleic acid and/or protein expression as well as CDAP activity, in the context of a biological sample (e.g., blood, serum, cells, or tissue) to thereby determine whether a subject is afflicted with a disease or disorder, or is at risk of developing a disorder, associated with aberrant CDAP expression or activity. The invention also provides for prognostic (or predictive) assays for determining whether an individual is at risk of developing a disorder associated with CDAP protein, nucleic acid expression or activity. For example, mutations in a CDAP gene can be assayed in a biological sample. Such assays can be used for prognostic or predictive purposes to, thereby, phophylactically treat a subject prior to the onset of a disorder characterized by or associated with a CDAP protein, nucleic acid expression or activity.

Another aspect of the invention pertains to monitoring the influence of agents (e.g., drugs, compounds) on the expression or activity of CDAP in clinical trials.

These and other agents are described in further detail in the following sections.

1. Diagnostic Assays

An exemplary method for detecting the presence or absence of CDAP nucleic acid or protein in a biological sample involves obtaining a biological sample from a test subject and contacting the biological sample with a compound or an agent capable of detecting CDAP nucleic acid or protein (e.g., MnRNA, genomic DNA) such that the presence of CDAP nucleic acid or protein is detected in the biological sample. A preferred agent for detecting CDAP MRNA or genomic DNA is a labeled nucleic acid probe capable of hybridizing to CDAP mRNA or genomic DNA. The nucleic acid probe can be, for example, a full-length CDAP nucleic acid, such as the nucleic acid of SEQ ID NO:1, 2, 3, 4, 5, or 7, or a portion thereof, such as an oligonucleotide of at least 15, 20, 25, 30, 35, 40, 45, 50, 100, 250 or 500 nucleotides in length and sufficient to specifically hybridize under stringent conditions to CDAP mRNA or genomic DNA. Other suitable probes for use in the diagnostic assays of the invention are described herein.

A preferred agent for detecting CDAP protein is an antibody capable of binding to CDAP protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect CDAP mRNA, genomic DNA, or protein in a biological sample in vitro as well as in vivo. For example, in vitro techniques for detection of CDAP mRNA include Northern hybridizations and in situ hybridizations. In vitro techniques for detection of CDAP protein include enzyme linked immunosorbent assays (ELISAs), Western blots, immunoprecipitations and immunofluorescence. In vitro techniques for detection of CDAP genomic DNA include Southern hybridizations. Furthermore, in vivo techniques for detection of CDAP protein include introducing into a subject a labeled anti-CDAP antibody. For example, the antibody can be labeled with a radioactive marker whose presence and location in a subject can be detected by standard imaging techniques.

In one embodiment, the biological sample contains protein molecules from the test subject. Alternatively, the biological sample can contain MRNA molecules from the test subject or genomic DNA molecules from the test subject. A preferred biological sample is a serum sample isolated by conventional means from a subject.

In another embodiment, the methods fuirther involve obtaining a control biological sample from a control subject, contacting the control sample with a compound or agent capable of detecting CDAP MRNA, genomic DNA, or protein, such that the presence of CDAP MRNA genomic DNA or protein is detected in the biological sample, and comparing the presence of CDAP MRNA, genomic DNA, or protein in the control sample with the presence of CDAP MRNA, genomic DNA, or protein in the test sample.

The invention also encompasses kits for detecting the presence of CDAP in a biological sample. For example, the kit can comprise a labeled compound or agent capable of detecting CDAP DNA, mRNA, or protein in a biological sample; means for determining the amount of CDAP in the sample; and means for comparing the amount of CDAP in the sample with a standard. The compound or agent can be packaged in a suitable container. The kit can further comprise instructions for using the kit to detect CDAP nucleic acid or protein.

2. Prognostic Assays

The diagnostic methods described herein can furthermore be utilized to identify subjects having or at risk of developing a disease or disorder associated with aberrant CDAP expression or activity. For example, the assays described herein, such as the preceding diagnostic assays or the following assays, can be utilized to identify a subject having or at risk of developing a disorder associated with a misregulation in CDAP protein activity or nucleic acid expression, such as a disorder characterized by deregulated programmed cell death, e.g., a neurodegenerative disorder. Thus, the present invention provides a method for identifying a disease or disorder associated with aberrant CDAP expression or activity, in which a test sample is obtained from a subject and CDAP nucleic acid (e.g., mRNA or genomic DNA) or protein is detected, wherein the presence of CDAP protein or nucleic acid is diagnostic for a subject having or at risk of developing a disease or disorder associated with aberrant CDAP expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant CDAP expression or activity. For example, such methods can be used to determine whether a subject can be effectively treated with an agent for a disorder characterized by deregulated programmed cell death, e.g., a neurodegenerative disorder. Thus, the present invention provides methods for determining whether a subject can be effectively treated with an agent for a disorder associated with aberrant CDAP expression or activity in which a test sample is obtained and CDAP protein or nucleic acid expression or activity is detected (e.g., wherein the abundance of CDAP nucleic acid or protein expression or activity is diagnostic for a subject that can be administered the agent to treat a disorder associated with aberrant CDAP expression or activity).

The methods of the invention can also be used to detect genetic alterations in a CDAP gene, thereby determining if a subject with the altered gene is at risk for a disorder characterized by misregulation in CDAP protein activity or nucleic acid expression, such as a neurodegenerative disorder. In preferred embodiments, the methods include detecting, in a sample of cells from the subject, the presence or absence of a genetic alteration characterized by at least one of an alteration affecting the integrity of a gene encoding a CDAP-protein, or the mis-expression of the CDAP gene. For example, such genetic alterations can be detected by ascertaining the existence of at least one of 1) a deletion of one or more nucleotides from a CDAP gene; 2) an addition of one or more nucleotides to a CDAP gene; 3) a substitution of one or more nucleotides of a CDAP gene, 4) a chromosomal rearrangement of a CDAP gene; 5) an alteration in the level of a messenger RNA transcript of a CDAP gene, 6) aberrant modification of a CDAP gene, such as of the methylation pattern of the genomic DNA, 7) the presence of a non-wild type splicing pattern of a messenger RNA transcript of a CDAP gene, 8) a non-wild type level of a CDAP-protein, 9) allelic loss of a CDAP gene, and 10) inappropriate post-translational modification of a CDAP-protein. As described herein, there are a large number of assays known in the art which can be used for detecting alterations in a CDAP gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the CDAP-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a CDAP gene under conditions such that hybridization and amplification of the CDAP-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a CDAP gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in CDAP can be identified by hybridizing a sample and control nucleic acids, e.g., DNA or RNA, to high density arrays containing hundreds or thousands of oligonucleotides probes (Cronin, M. T. et al. (1996) Human Mutation 7:244-255; Kozal, M. J. et al. (1996) Nature Medicine 2:753-759). For example, genetic mutations in CDAP can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CDAP gene and detect mutations by comparing the sequence of the sample CDAP with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the CDAP gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type CDAP sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in CDAP cDNAs obtained from samples of cells. For example, the mut Y enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a CDAP sequence, e.g., a wild-type CDAP sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in CDAP genes. For example, single strand conformation polymorphism (SSCP) may be used to detect differences in electrophoretic mobility between mutant and wild type nucleic acids (orita et al. (1989) Proc Natl. Acad. Sci USA: 86:2766, see also Cotton (1993) Mutat. Res. 285:125-144; and Hayashi (1992) Genet. AnaL Tech. Appl. 9:73-79). Single-stranded DNA fragments of sample and control CDAP nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a CDAP gene.

Furthermore, any cell type or tissue in which CDAP is expressed may be utilized in the prognostic assays described herein.

3. Monitoring of Effects During Clinical Trials

Monitoring the influence of agents (e.g., drugs) on the expression or activity of a CDAP protein can be applied not only in basic drug screening, but also in clinical trials. For example, the effectiveness of an agent determined by a screening assay as described herein to decrease CDAP gene expression, protein levels, or downregulate CDAP activity, can be monitored in clinical trials of subjects exhibiting increased CDAP gene expression protein levels, or upregulated CDAP activity. Alternatively, the effectiveness of an agent determined by a screening assay to increase CDAP gene expression, protein levels, or upregulate CDAP activity, can be monitored in clinical trials of subjects exhibiting decreased CDAP gene expression, protein levels, or downregulated CDAP activity. In such clinical trials, the expression or activity of a CDAP gene, and preferably, other genes that have been implicated in, for example, a CDAP-associated disorder can be used as a “read out” or markers of the phenotype of a particular cell.

For example, and not by way of limitation, genes, including CDAP, that are 5 modulated in cells by treatment with an agent (e.g., compound, drug or small molecule) which modulates CDAP activity (e.g., identified in a screening assay as described herein) can be identified. Thus, to study the effect of agents on CDAP-associated disorders (e.g., disorders characterized by deregulated programmed cell death), for example, in a clinical trial, cells can be isolated and RNA prepared and analyzed for the levels of expression of CDAP and other genes implicated in the CDAP-associated disorder, respectively. The levels of gene expression (e.g., a gene expression pattern) can be quantified by northern blot analysis or RT-PCR, as described herein, or alternatively by measuring the amount of protein produced, by one of the methods described herein, or by measuring the levels of activity of CDAP or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a CDAP mRNA, genomic DNA, or protein in the pre-administration sample; (iii) obtaining one or more post-administration samples from the subject; (iv) detecting the level of expression or activity of the CDAP mRNA, genomic DNA, or protein in the post-administration samples; (v) comparing the level of expression or activity of the CDAP mRNA, genomic DNA, or protein in the pre-adm-inistration sample with the CDAP mRNA, genomic DNA, or protein in the post administration sample or samples; and (vi) altering the administration of the agent to the subject accordingly. For example, increased administration of the agent may be desirable to decrease the expression or activity of CDAP to lower levels than detected, i.e., to increase the effectiveness of the agent. Alternatively, decreased administration of the agent may be desirable to increase expression or activity of CDAP to higher levels than detected, i.e. to decrease the effectiveness of the agent. According to such an embodiment, CDAP expression or activity may be used as an indicator of the effectiveness of an agent, even in the absence of an observable phenotypic response.

B. Methods of Treatment:

The present invention provides for both prophylactic and therapeutic methods of treating a subject at risk of (or susceptible to) or having a disorder associated with aberrant CDAP expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the CDAP molecules of the present invention or CDAP modulators according to that individual's drug response genotype. Pharmacogenomics allow a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant CDAP expression or activity, by administering to the subject a CDAP or an agent which modulates CDAP expression or at least one CDAP activity. Subjects at risk for a disease which is caused or contributed to by aberrant CDAP expression or activity can be identified by, for example, any or a combination of diagnostic or prognostic assays as described herein. Administration of a prophylactic agent can occur prior to the manifestation of symptoms characteristic of the CDAP aberrancy, such that a disease or disorder is prevented or, alternatively, delayed in its progression. Depending on the type of CDAP aberrancy, for example, a CDAP molecule, CDAP agonist or CDAP antagonist can be used for treating the subject. The appropriate agent can be determined based on screening assays described herein.

2. Therapeutic Methods

Another aspect of the invention pertains to methods of modulating CDAP expression or activity for therapeutic purposes. Accordingly, in an exemplary embodiment, the modulatory method of the invention involves contacting a cell with a CDAP molecule or an agent which modulates (e.g., upregulates or downregulates) CDAP expression or activity. In another embodiment, the method involves administering a CDAP protein or nucleic acid molecule as therapy to compensate for reduced or aberrant CDAP expression or activity.

In another embodiment, the agent inhibits one or more CDAP activities. Examples of such inhibitory agents include antisense CDAP nucleic acid molecules, anti-CDAP antibodies, and CDAP inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a CDAP protein or nucleic acid molecule.

Stimulation of CDAP activity is desirable in situations in which CDAP is abnormally downregulated and/or in which increased CDAP activity is likely to have a beneficial effect. For example, stimulation of CDAP activity is desirable in situations in which increased CDAP activity is likely to have a beneficial effect, e.g., in the case a proliferative disorder. Likewise, inhibition of CDAP activity is desirable in situations in which CDAP is abnormally upregulated and/or in which decreased CDAP activity is likely to have a beneficial effect, e.g., in the case of a neurodegenerative disorder.

In yet another embodiment, the modulatory method of the invention involves contacting a cell with a CDAP molecule or an agent which modulates one or more of the activities of CDAP protein activity associated with the cell. An agent that modulates CDAP protein activity can be an agent as described herein, such as a nucleic acid or a protein, a naturally-occurring target molecule of a CDAP protein (e.g., a CDAP substrate), a CDAP antibody, a CDAP agonist or antagonist, a peptidomimetic of a CDAP agonist or antagonist, or other small molecule. In one embodiment, the agent stimulates one or more CDAP activities. Examples of such stimulatory agents include active CDAP protein and a nucleic acid molecule encoding CDAP that has been introduced into the cell.

3. Pharmacogenomics

The CDAP molecules of the present invention, as well as agents, or modulators which have a stimulatory or inhibitory effect on CDAP activity (e.g., CDAP gene expression) as identified by a screening assay described herein can be administered to individuals to treat (prophylactically or therapeutically) disorders associated with abberant CDAP activity (e.g., of disorders characterized by deregulated programmed cell death such as neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementias; myelodysplastic syndromes, e.g., aplastic anemia; ischemic injury, e.g., myocardial infarction, stroke, or reperfusion injury; autommune disorders, e.g., systemic lupus erythematosus, or immune-mediated glomerulonephritis; or profilerative disorders, e.g., cancer such as follicular lymphomas, carcinomas with p53 mutations, or hormone-dependent tumors, e.g., breast cancer, prostate cancer, or ovarian cancer). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a CDAP molecule or CDAP modulator as well as tailoring the dosage and/or therapeutic regimen of treatment with a CDAP molecule or CDAP modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a CDAP protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a CDAP molecule or CDAP modulator of the present invention) can give an indication whether gene pathways related to toxicity have been turned on.

Inform,ation generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a CDAP molecule or CDAP modulator, such as a modulator identified by one of the exemplary screening assays described herein.

C. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the CDAP nucleotide sequences, described herein, can be used to map the location of the CDAP genes on a chromosome. The mapping of the CDAP sequences to chromosomes is an important first step in correlating these sequences with genes associated with disease.

Briefly, CDAP genes can be mapped to chromosomes by preparing PCR primers (preferably 15-25 bp in length) from the CDAP nucleotide sequences. Computer analysis of the CDAP sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the CDAP sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the CDAP nucleotide sequences to design oligonucleotide primers, sublocalization can be achieved with panels of fragments from specific chromosomes. Other mapping strategies which can similarly be used to map a CDAP sequence to its chromosome include in situ hybridization (described in Fan, Y. et al. (1990) Proc. NatiL Acad. Sci. USA, 87:6223-27), pre-screening with labeled flQw-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the-DNA sequences between individuals affected and unaffected with a disease associated with the CDAP gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The CDAP sequences of the present invention can also be used to identify individuals from minute biological samples. The United States military, for example, is considering the use of restriction fragment 4ength polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. 5,272,057).

Furthermore, the sequences of the present invention can be used to provide an alternative technique which determines the actual base-by-base DNA sequence of selected portions of an individual's genome. Thus, the CDAP nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The CDAP nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 2, 3, 4, 5, or 7, can comfortably provide positive individual identification with a panel of perhaps 10 to 1,000 primers which each yield a noncoding amplified sequence of 100 bases.

If a panel of reagents from CDAP nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial CDAP Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1, 2, 3, 4, 5, or 7 are particularly appropriate for this use as greater numbers of polymorphisms occur in the noncoding regions, making it easier to differentiate individuals using this technique. Examples of polynucleotide reagents include the CDAP nucleotide sequences or portions thereof, e.g., fragments derived from the noncoding regions of SEQ ID NO: 1, 2, 3, 4, 5, or 7, having a length of at least 20 bases, preferably at least 30 bases.

The CDAP nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such CDAP probes can be used to identify tissue by species and/or by organ type.

In a similar fashion, these reagents, e.g., CDAP primers or probes can be used to screen tissue culture for contamination (i.e. screen for the presence of a mixture of different types of cells in a culture).

D. Screening Assays:

The invention provides a method (also referred to herein as a “screening assay”) for identifying modulators, e.g., candidate or test compounds or agents (e.g., peptides, peptidomimetics, small molecules, or other drugs) which bind to CDAP proteins, have a stimulatory or inhibitory effect on, for example, CDAP expression or CDAP activity, or have a stimulatory or inhibitory effect on, for example, the expression or activity of a CDAP substrate.

In one embodiment, the invention provides assays for screening candidate or test compounds which are substrates of a CDAP protein or polypeptide or biologically active portion thereof. In another embodiment, the invention provides assays for screening candidate or test compounds which bind to or modulate the activity of a CDAP protein or polypeptide or biologically active portions thereof. The test compounds of the present invention can be obtained using any of the numerous approaches in combinatorial library methods known in the art, including: biological libraries; spatially addressable parallel solid phase or solution phase libraries; synthetic.library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the syntesis of molecular libraries can be found in, for example, DeWitt et al. (1993) Proc. Natl. Acad. Sci. U.S.A. 90:6909; Erb et al. (1994) Proc. Natl. Acad. Sci. USA 91:11422; Zuckermaun et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner USP 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a CDAP protein or biologically active portion thereof is contacted with a test compound and the ability of the test compound to modulate CDAP activity is determined. Determining the ability of the test compound to modulate CDAP activity can be accomplished by monitoring, for example, the survival of a cell which expresses CDAP. The cell, for example, can be of mammalian origin. The ability of the test compound to modulate the ability of CDAP to bind to a substrate can also be determined by, for example, coupling the CDAP substrate with a radioisotope or enzymatic label such that binding of the CDAP substrate to CDAP can be determined by detecting the labeled CDAP substrate in a complex. For example, compounds (e.g., CDAP substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a CDAP substrate) to interact with CDAP without the labeling of any of the interactants. For example, a microphysiometer can be used to detect the interaction of a compound with CDAP without the labeling of either the compound or the CDAP. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and CDAP.

In another preferred embodiment, the assay comprises contacting a cell which is responsive to a CDAP protein or biologically active portion thereof, with a CDAP protein or biologically-active portion thereof, to form an assay mixture, contacting the assay mixture with a test compound, and determining the ability of the test compound to modulate the activity of the CDAP protein or biologically active portion thereof, wherein determining the ability of the test compound to modulate the activity of the CDAP protein or biologically active portion thereof comprises determining the ability of the test compound to modulate a biological activity of the CDAP-responsive cell (e.g., determining the ability of the test compound to modulate programmed cell death in the cell).

In another embodiment, an assay is a cell-based assay comprising contacting a cell expressing a CDAP target molecule (e.g., a CDAP substrate) with a test compound and determining the ability of the test compound to modulate (e.g. stimulate or inhibit) the activity of the CDAP target molecule. Determining the ability of the test compound to modulate the activity of a CDAP target molecule can be accomplished, for example, by determining the ability of the CDAP protein to bind to or interact with the CDAP target molecule.

Determining the ability of the CDAP protein to bind to or interact with a CDAP target molecule can be accomplished by one of the methods described above for determining direct binding. In a preferred embodiment, determining the ability of the CDAP protein to bind to or interact with a CDAP target molecule can be accomplished by determining the activity of the target molecule. For example, the activity of the target molecule can be determined by detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a CDAP protein or biologically active portion thereof is contacted with a test compound and the ability of the test compound to bind to the CDAP protein or biologically active portion thereof is determined. Binding of the test compound to the CDAP protein can be determined either directly or indirectly as described above. In a preferred embodiment, the assay includes contacting the CDAP protein or biologically active portion thereof with a known compound which binds CDAP to form an assay mixture, contacting the assay mixture with a test compound, and determining the ability of the test compound to interact with a CDAP protein, wherein determining the ability of the test compound to interact with a CDAP protein comprises determining the ability of the test compound to preferentially bind to CDAP or biologically active portion thereof as compared to the known compound.

In another embodiment, the assay is a cell-free assay in which a CDAP protein or biologically active portion thereof is contacted with a test compound and the ability of the test compound to modulate (e.g., stimulate or inhibit) the activity of the CDAP protein or biologically active portion thereof is determined. Determining the ability of the test compound to modulate the activity of a CDAP protein can be accomplished, for example, by determining the ability of the CDAP protein to bind to a CDAP target molecule by one of the methods described above for determining direct binding. Determining the ability of the CDAP protein to bind to a CDAP target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BlAcore). Changes in the optical phenomenon of surface plasmon resonance (SPR) can be used as an indication of real-time reactions between biological molecules.

In an alternative embodiment, determining the ability of the test compound to modulate the activity of a CDAP protein can be accomplished by determining the ability of the CDAP protein to further modulate the activity of a downstream effector of a CDAP target molecule. For example, the activity of the effector molecule on an appropriate target can be determined or the binding of the effector to an appropriate target can be determined as previously described.

In yet another embodiment, the cell-free assay involves contacting a CDAP protein or biologically active portion thereof with a known compound which binds the CDAP protein to form an assay mixture, contacting the assay mixture with a test compound, and determining the ability of the test compound to interact with the CDAP protein, wherein determining the ability of the test compound to interact with the CDAP protein comprises determining the ability of the CDAP protein to preferentially bind to or modulate the activity of a CDAP target molecule.

In more than one embodiment of the above assay methods, it may be desirable to immobilize either CDAP or its target molecule to facilitate separation of complexed from uncomplexed forms of one or both of the proteins, as well as to accommodate automation of the assay. Binding of a test compound to a CDAP protein, or interaction of a CDAP protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/CDAP fusion proteins or glutathione-S-transferase/target fusion proteins can be adsorbed onto glutathione sepharose beads (Sigma Chemical, St. Louis, Mo.) or- glutathione derivatized microtitre plates, which are then combined with the test compound or the test compound and either the non-adsorbed target protein or CDAP protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of CDAP binding or activity determined using standard techniques.

Other techniques for immobilizing proteins on matrices can also be used in the screening assays of the invention. For example, either a CDAP protein or a CDAP target molecule can be immobilized utilizing conjugation of biotin and streptavidin. Biotinylated CDAP protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with CDAP protein or target molecules but which do not interfere with binding of the CDAP protein to its target molecule can be derivatized to the wells of the plate, and unbound target or CDAP protein trapped in the wells by antibody conjugation. Methods for detecting such complexes, in addition to those described above for the GET-immobilized complexes, include immunodetection of complexes using antibodies reactive with the CDAP protein or target molecule, as well as enzyme-linked assays which rely on detecting an enzymatic activity associated with the CDAP protein or target molecule.

In another embodiment, modulators of CDAP expression are identified in a method wherein a cell is contacted with a candidate compound and the expression of CDAP mRNA or protein in the cell is determined. The level of expression of CDAP mRNA or protein in the presence of the candidate compound is compared to the level of expression of CDAP mRNA or protein in the absence of the candidate compound. The candidate compound can then be identified as a modulator of CDAP expression based on this comparison. For example, when expression of CDAP mRNA or protein is greater (statistically significantly greater) in the presence of the candidate compound than in its absence, the candidate compound is identified as a stimulator of CDAP mRNA or protein expression. 1 Alternatively, when expression of CDAP mRNA or protein is less (statistically significantly less) in the presence of the candidate compound than in its absence, the candidate compound is identified as an inhibitor of CDAP mRNA or protein expression. The level of CDAP mRNA or protein expression in the cells can be determined by methods described herein for detecting CDAP mRNA or protein.

In yet another aspect of the invention, the CDAP proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent W094/10300), to identify other proteins, which bind to or interact with CDAP (“CDAP-binding proteins” or “CDAP-bp”) and are involved in CDAP activity. Such CDAP-binding proteins are also likely to be involved in the propagation of signals by the CDAP proteins or CDAP targets as, for example, downstream elements of a CDAP-mediated signaling pathway. Alternatively, such CDAP-binding proteins are likely to be CDAP inhibitors.

The two-hybrid system is based on the modular nature of most transcription factors, which consist of separable DNA-binding and activation domains. Briefly, the assay utilizes two different DNA constructs. In one construct, the gene that codes for a CDAP protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CDAP-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the CDAP protein.

This invention further pertains to novel agents identified by the above-described screening assays. Accordingly, it is within the scope of this invention to further use an agent identified as described herein in an appropriate animal model. For example, an agent identified as described herein (e.g., a CDAP modulating agent, an antisense CDAP nucleic acid molecule, a CDAP-specific antibody, or a CDAP-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention, pertains to uses of novel agents identified by the above-described screening assays for treatments as described herein.

III. Recombinant Expression Vectors and Host Cells

Another aspect of the invention pertains to vectors, preferably expression vectors, containing a CDAP nucleic acid or a portion thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the nucleic acid molecule in a host cell. For example, the recombinant expression vectors can include one or more regulatory sequences, selected on the basis of the host cells to be used for expression, operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described in, for example, Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., CDAP proteins, mutant forms of CDAP proteins, fusion proteins, and the like).

The recombinant expression vectors of the invention can be designed for expression of CDAP proteins in prokaryotic or eukaryotic cells. For example, CDAP proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in CDAP activity assays, (e.g., direct assays or competitive assays described in detail below), or to, for example, generate antibodies specific for CDAP proteins. In a preferred embodiment, a CDAP fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

Examples of suitable inducible non-fusion E. coli expression vectors include pTrc (Amann et al., (1988) Gene 69:301-315) and pET 1Id (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11 d vector relies on transcription from a T7 gnlO-lac fusion promoter mediated by a coexpressed viral RNA polymerase (T7 gnl). This viral polymerase is supplied by host strains BL2I(DE3) or HMSI74(DE3) from a resident prophage harboring a T7 gnl gene under the transcriptional control of the lacUV 5 promoter.

One strategy to maximize recombinant protein expression in E. coli is to express the protein in a bacterial host with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the CDAP expression vector is a yeast expression vector. Examples of vectors for expression in yeast S. cerivisae include pYepSecl (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, CDAP proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufinan et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid molecule preferentially in a particular cell type (e.g., tissue-specific regulatory elements are used to express the nucleic acid molecule). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to CDAP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a CDAP protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory ManuaL 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G4 18, hygromycin and methotrexate. Nucleic acid encoding a selectable marker can be introduced into a host cell on the same vector as that encoding a CDAP protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a CDAP protein. Accordingly, the invention further provides methods for producing a CDAP protein using the host cells of the invention. In one embodiment, the method comprises culturing the host cell of invention (into which a recombinant expression vector encoding a CDAP protein has been introduced) in a suitable medium such that a CDAP protein is produced. In another embodiment, the method further comprises isolating a CDAP protein from the medium or the host cell.

The host cells of the invention can also be used to produce non-human transgenic animals. For example, in one embodiment, a host cell of the invention is a fertilized oocyte or an embryonic stem cell into which CDAP-coding sequences have been introduced. Such host cells can then be used to create non-human transgenic animals in which exogenous CDAP sequences have been introduced into their genome or homologous recombinant animals in which endogenous CDAP sequences have been altered. Such animals are useful for studying the function and/or activity of a CDAP and for identifying and/or evaluating modulators of CDAP activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous CDAP gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a CDAP-encoding nucleic acid into the male pronuclei of a fertilized oocyte, e.g., by microinjection, retroviral infection, and allowing the oocyte to develop in a pseudopregnant female foster animal. The CDAP cDNA sequence of SEQ ID NO: 1, 2, 3, 4, 5, or 7 can be introduced as a transgene into the genome of a non-human animal. Alternatively, a nonhuman homologue of a human CDAP gene, such as a mouse or rat CDAP gene, can be used as a transgene. Alternatively, a CDAP gene homologue, such as another CDAP family member, can be isolated based on hybridization to the CDAP cDNA sequences of SEQ ID NO:1, 2, 3, 4, 5, or 7 or the DNA insert of the plasmid deposited with ATCC as Accession Number or (described further in subsection 1 above) and used as a transgene. Intronic sequences and polyadenylation signals can also be included in the transgene to increase the efficiency of expression of the transgene. A tissue-specific regulatory sequence can be operably linked to a CDAP transgene to direct expression of a CDAP protein to particular cells. Methods for generating transgenic animals via embryo manipulation and microinjection, particularly animals such as mice, are described in, for example, U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a CDAP transgene in its genome and/or expression of CDAP mRNA in tissues or cells of the animals. A transgenic founder animal can then be used to breed additional animals carrying the transgene. Moreover, transgenic animals carrying a transgene encoding a CDAP protein can further be bred to other transgenic animals carrying other transgenes.

To create a homologous recombinant animal, a vector is prepared which contains at least a portion of a CDAP gene into which a deletion, addition or substitution has been introduced to thereby alter, e.g., functionally disrupt, the CDAP gene. The CDAP gene can be a human gene (e.g., a cDNA isolated by stringent hybridization with the nucleotide sequence of SEQ ID NO:1, 2, 3, 4, 5, or 7), but more preferably, is a non-human homologue of a human CDAP gene (e.g., the cDNA of SEQ ID NO:1, 2, 3, 4, 5, or 7). For example, a mouse CDAP gene can be used to construct a homologous recombination vector suitable for altering an endogenous CDAP gene in the mouse genome. In a preferred embodiment, the vector is designed such that, upon homologous recombination, the endogenous CDAP gene is functionally disrupted (i.e., no longer encodes a fimctional protein; also referred to as a “knock out” vector). The vector can also be designed such that, upon homologous recombination, the endogenous CDAP gene is mutated or otherwise altered but still encodes a functional protein (e.g., the upstream regulatory region can be altered to thereby alter the expression of the endogenous CDAP protein). In the homologous recombination vector, the altered portion of the CDAP gene is flanked at its 5′ and 3′ ends by additional nucleic acid sequence of the CDAP gene to allow for homologous recombination to occur between the exogenous CDAP gene carried by the vector and an endogenous CDAP gene in an embryonic stem cell. The additional flanking CDAP nucleic acid sequence is of sufficient length for successful homologous recombination with the endogenous gene. Typically, several kilobases of flanking DNA (both at the 5′ and 3′ ends) are included in the vector (see, e.g., Thomas, K. R. and Capecchi, M. R. (1987) Cell 51:503 for a description of homologous recombination vectors). The vector is introduced into an embryonic stem cell line (e.g., by electroporation) and cells in which the introduced CDAP gene has homologously recombined with the endogenous CDAP gene are selected (see, e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Bems et al.

In another embodiment, transgenic non-human animals can be produced which contain selected systems which allow for regulated expression of the transgene. One example of such a system is the cre/loxP recombinase system of bacteriophage P1. For a description of the crelloxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The recontructed oocyte is then cultured such that it develops to a morula or a blastocyte and then transferred to a pseudopregnant female foster animal. The offspring borne of this female foster animal will be clones of the animal from which the cell, e.g., the somatic cell, is isolated.

IV. Pharmaceutical Compositions

The CDAP nucleic acid molecules, CDAP proteins, and anti-CDAP antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenedicminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifumgal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a CDAP nucleic acid molecule a CDAP protein, or anti-CDAP antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD501ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein.

When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Isolated CDAP Proteins and Anti-CDAP Antibodies

One aspect of the invention pertains to isolated CDAP proteins, and biologically active portions thereof, as well as polypeptide fragments suitable for use as immunogens to raise anti-CDAP antibodies. In one embodiment, native CDAP proteins can be isolated from cells or tissue sources by an appropriate purification scheme using standard protein purification techniques. In another embodiment, CDAP proteins are produced by recombinant DNA techniques. Alternative to recombinant expression, a CDAP protein or polypeptide can be synthesized chemically using standard peptide synthesis techniques.

An “isolated” or “purified” protein or biologically active portion thereof is substantially free of cellular material or other contaminating proteins from the cell or tissue source from which the CDAP protein is derived, or substantially free from chemical precursors or other chemicals when chemically synthesized. The language “substantially free of cellular material” includes preparations of CDAP protein in which the protein is separated from cellular components of the cells from which it is isolated or recombinantly produced. In one embodiment, the language “substantially free of cellular material” includes preparations of CDAP protein having less than about 30% (by dry weight) of non-CDAP protein (also referred to herein as a “contaminating protein”), more preferably less than about 20% of non-CDAP protein, still more preferably less than about 10% of non-CDAP protein, and most preferably less than about 5% non-CDAP protein. When the CDAP protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CDAP protein in which the protein is separated from chemical precursors or other chemicals which are involved in the synthesis of the protein. In one embodiment, the language “substantially free of chemical precursors or other chemicals” includes preparations of CDAP protein having less than about 30% (by dry weight) of chemical precursors or non-CDAP chemicals, more preferably less than about 20% chemical precursors or non-CDAP chemicals, still more preferably less than about 10% chemical precursors or non-CDAP chemicals, and most preferably less than about 5% chemical precursors or non-CDAP chemicals.

Biologically active portions of a CDAP protein include peptides comprising amino acid sequences sufficiently homologous to or derived from the amino acid sequence of the CDAP protein, e.g., the amino acid sequence shown in SEQ ID NO:6, which include less amino acids than the full length CDAP proteins, and exhibit at least one activity of a CDAP protein. Typically, biologically active portions comprise a domain or motif with at least one activity of the CDAP protein. A biologically active portion of a CDAP protein. can be a polypeptide which is, for example, 10, 25, 50, 100, 200 or more amino acids in length.

In a preferred embodiment, the CDAP protein has an amino acid sequence shown in SEQ ID NO:6. In other embodiments, the CDAP protein is substantially homologous to SEQ ID NO:6, and retains the functional activity of the protein of SEQ ID NO:6, yet differs in amino acid sequence due to natural allelic variation or mutagenesis, as described in detail in subsection 1 above. Accordingly, in another embodiment, the CDAP protein is a protein which comprises an amino acid sequence at least about 30%, 35%, 40%, 45%, 50%, 55%, 60%, 65%, 70%, 75%, 80%, 85%, 90%, 95% 98% or more homologous to SEQ ID NO:6.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to CDAP nucleic acid molecules of the invention. BLAST protein searches can be performed with the XBLAST program, score=50, wordlength=3 to obtain amino acid sequences homologous to CDAP protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides CDAP chimeric or fusion proteins. As used herein, a CDAP “chimeric protein” or “fusion protein” comprises a CDAP polypeptide operatively linked to a non-CDAP polypeptide. An “CDAP polypeptide” refers to a polypeptide having an amino acid sequence corresponding to CDAP, whereas a “non-CDAP polypeptide” refers to a polypeptide having an amino acid sequence corresponding to a protein which is not substantially homologous to the CDAP protein, e.g., a protein which is different from the CDAP protein and which is derived from the same or a different organism. Within a CDAP fusion protein the CDAP polypeptide can correspond to all or a portion of a CDAP protein. In a preferred embodiment, a CDAP fusion protein comprises at least one biologically active portion of a CDAP protein. In another preferred embodiment, a CDAP fusion protein comprises at least two biologically active portions of a CDAP protein. Within the fusion protein, the term “operatively linked” is intended to indicate that the CDAP polypeptide and the non-CDAP polypeptide are fused in-frame to each other. The non-CDAP polypeptide can be fused to the N-terminus or C-terminus of the CDAP polypeptide.

For example, in one embodiment, the fusion protein is a GST-CDAP fusion protein in which the CDAP sequences are fused to the C-terminus of the GST sequences. Such fusion proteins can facilitate the purification of recombinant CDAP.

In another embodiment, the fusion protein is a CDAP protein containing a heterologous signal sequence at its N-terminus. In certain host cells (e.g., mammalian host cells), expression and/or secretion of CDAP can be increased through use of a heterologous signal sequence.

The CDAP fusion proteins of the invention can be incorporated into pharmaceutical compositions and administered to a subject in vivo. The CDAP fusion proteins can be used to affect the bioavailability of a CDAP substrate. Use of CDAP fusion proteins may be useful therapeutically for the treatment of disorders characterized by deregulated programmed cell such as neurodegenerative disorders, e.g., Alzheimer's disease, dementias related to Alzheimer's disease (such as Pick's disease), Parkinson's and other Lewy diffuse body diseases, multiple sclerosis, amyotrophic lateral sclerosis, progressive supranuclear palsy, epilepsy, Jakob-Creutzfieldt disease, or AIDS related dementias; myelodysplastic syndromes, e.g., aplastic anemia; ischemic injury, e.g., myocardial infarction, stroke, or reperfusion injury; autommune disorders, e.g., systemic lupus erythematosus, or immune-mediated glomerulonephritis; or profilerative disorders, e.g., cancer.

Moreover, the CDAP-fusion proteins of the invention can be used as immunogens to produce anti-CDAP antibodies in a subject, to purify CDAP ligands and in screening assays to identify molecules which inhibit the interaction of CDAP with a CDAP substrate.

Preferably, a CDAP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A CDAP-encoding nucleic acid can be cloned into such an expression vector such that the fusion moiety is linked in-frame to the CDAP protein.

The present invention also pertains to variants of the CDAP proteins which function as either CDAP agonists (mimetics) or as CDAP antagonists. Variants of the CDAP proteins can be generated by mutagenesis, e.g., discrete point mutation or truncation of a CDAP protein. An agonist of the CDAP proteins can retain substantially the same, or a subset, of the biological activities of the naturally occurring form of a CDAP protein. An antagonist of a CDAP protein can inhibit one or more of the activities of the naturally occurring form of the CDAP protein by, for example, competitively modulating programmed cell death associated activity of a CDAP protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the CDAP protein.

In one embodiment, variants of a CDAP protein which function as either CDAP agonists (mimetics) or as CDAP antagonists can be identified by screening combinatorial libraries of mutants, e.g., truncation mutants, of a CDAP protein for CDAP protein agonist or antagonist activity. In one embodiment, a variegated library of CDAP variants is generated by combinatorial mutagenesis at the nucleic acid level and is encoded by a variegated gene library. A variegated library of CDAP variants can be produced by, for example, enzymatically ligating a mixture of synthetic oligonucleotides into gene sequences such that a degenerate set of potential CDAP sequences is expressible as individual polypeptides, or alternatively, as a set of larger fusion proteins (e.g., for phage display) containing the set of CDAP sequences therein. There are a variety of methods which can be used to produce libraries of potential CDAP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CDAP sequences. Methods for synthesizing degenerate oligonucleotides are known in the art (see, e.g., Narang, S.A. (1983) Tetrahedron 39:3; Itakura et al. (1984) Annu. Rev. Biochem. 53:323; Itakura et al. (1984) Science 198:1056; Ice et al. (1983) Nucleic Acid Res. 11:477.

In addition, libraries of fragments of a CDAP protein coding sequence can be used to generate a variegated population of CDAP fragments for screening and subsequent selection of variants of a CDAP protein. In one embodiment, a library of coding sequence fragments can be generated by treating a double stranded PCR fragment of a CDAP coding sequence with a nuclease under conditions wherein nicking occurs only about once per molecule, denaturing the double stranded DNA, reentering the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the CDAP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CDAP proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify CDAP variants (Arkin and Yourvan (1992) Proc. Natl. Acad. Sci. USA 89:7811-7815; Delgrave et al. (1993) Protein Engineering 6(3):327-33 1).

In one embodiment, cell based assays can be exploited to analyze a variegated CDAP library. For example, a library of expression vectors can be transfected into a cell line which ordinarily synthesizes CDAP. The transfected cells can then be cultured such that CDAP and a particular mutant CDAP are expressed and the effect of expression of the mutant on CDAP activity in the cells can be detected, e.g., by any of a number of enzymatic assays or by monitoring cell survival, e.g., by monitoring cellular morphological features such as chromatin condensation. Plasmid DNA can then be recovered from the cells which score for inhibition, or alternatively, potentiation of CDAP activity, and the individual clones further characterized.

In another embodiment, a variegated CDAP library can be analyzed by determining the ability of the CDAP variants to modulate programmed cell death in a tissue which normally expresses CDAP, e.g., neuron. Neuronal survival can be determined by the MTT assay, as described in, for example, Kubo, T., et al., (1985) Dev. Brain Res., 85:249-258, the contents of which are incorporated herein by reference. Briefly, the tetrazolium salt 3-(4,5-dimethyl-2-thiazolyl-2,5-diphenyl-2Htetrazolium bromide (MTT) can be added to the neuronal culture medium at a final concentration of 1 mg/ml. After incubation at 37° C. for 2 h, the assay can be stopped with lysis buffer consisting of 20% (w/v) sodium dodecyl sulfate (SDS) and 50% (v/v) sodium N,N-dimethyl formamide, pH 4.7. The absorbance is measured photometrically at 570 nm after an incubation at 37° C. overnight.

An isolated CDAP protein, or a portion or fragment thereof, can be used as an immunogen to generate antibodies that bind CDAP using standard techniques for polyclonal and monoclonal antibody preparation. A full-length CDAP protein or antigenic peptide fragments of CDAP can be used as immunogens. An antigenic peptide of CDAP comprises at least 8 amino acid residues of the amino acid sequence shown in SEQ ID NO:6 and encompasses an epitope of CDAP such that an antibody raised against the peptide forms a specific immune complex with CDAP. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of CDAP that are located on the surface of the protein, e.g., hydrophilic regions.

A CDAP immunogen typically is used to prepare antibodies by immunizing a suitable subject, (e.g., rabbit, goat, mouse or other mammal) with the immunogen. An appropriate immunogenic preparation can contain, for example, recombinantly expressed CDAP protein or a chemically synthesized CDAP polypeptide. The preparation can further include an adjuvant, such as Freund's complete or incomplete adjuvant, or similar immunostimulatory agent. Immunization of a suitable subject with an immunogenic CDAP preparation induces a polyclonal anti-CDAP antibody response.

Accordingly, another aspect of the invention pertains to anti-CDAP antibodies. The term “antibody” as used herein refers to immunoglobulin molecules and immunologically active portions of immunoglobulin molecules, i.e., molecules that contain an antigen binding site which specifically binds (immunoreacts with) an antigen, such as CDAP. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind CDAP. The term “monoclonal antibody” or “monoclonal antibody composition”, as used herein, refers to a population of antibody molecules that contain only one species of an antigen binding site capable of immunoreacting with a particular epitope of CDAP. A monoclonal antibody composition, thus, typically displays a single binding affinity for a particular CDAP protein with which it immunoreacts.

Polyclonal anti-CDAP antibodies can be prepared as described above by immunizing a suitable subject with a CDAP immunogen. The anti-CDAP antibody titer in the immunized subject can be monitored over time by standard techniques, such as with an enzyme linked immunosorbent assay (ELISA) using immobilized CDAP. If desired, the antibody molecules directed against CDAP can be isolated from the mammal (e.g., from the blood) and further purified by well known techniques, such as protein A chromatography to obtain the IgG fraction. At an appropriate time after immunization, e.g., when the anti-CDAP antibody titers are the highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a CDAP immunogen as described above, and the culture supernatants of the resulting hybridoma cells are screened to identify a hybridoma producing a monoclonal antibody that binds CDAP.

Any of the many well known protocols used for fusing lymphocytes and immortalized cell lines can be applied for the purpose of generating an anti-CDAP monoclonal antibody (see, e.g., G. Galfre et al. (1977) Nature 266:55052; Gefter et al. Somatic Cell Genet., cited supra; Lerner, Yale J Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Agl4 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind CDAP, e.g., using a standard ELISA assay.

Alternative to preparing monoclonal antibody-secreting hybridomas, a monoclonal anti-CDAP antibody can be identified and isolated by screening a recombinant combinatorial immunoglobulin library (e.g., an antibody phage display library) with CDAP to thereby isolate immunoglobulin library members that bind CDAP. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be fourd in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (19,89) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-CDAP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. 5,225,539; Jones et al. (1986) Nature 321:552-525; Verhoeyan et al. (1988) Science 239:1534; and Beidler et al. (1988) J. Immunol. 141:4053-4060.

An anti-CDAP antibody (e.g., monoclonal antibody) can be used to isolate CDAP by standard techniques, such as affinity chromatography or immunoprecipitation. An anti-CDAP antibody can facilitate the purification of natural CDAP from cells and of recombinantly produced CDAP expressed in host cells. Moreover, an anti-CDAP antibody can be used to detect CDAP protein (e.g., in a cellular lysate or cell supernatant) in order to evaluate the abundance and pattern of expression of the CDAP protein. Anti-CDAP antibodies can be used diagnostically to monitor protein levels in tissue as part of a clinical testing procedure to, for example, determine the efficacy of a given treatment regimen. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

IDENTIFICATION AND CHARACTERIZATION OF CDAP cDNA

In this example, the identification and characterization of the genes encoding rat CDAP-1, CDAP-2, CDAP-3, CDAP-4, CDAP-5, and CDAP-6 (also referred to as rlrx-006, rlrx-013, rlrx-015, rlrx-018, rlrx-020, and rlrx-022, respectively) are described.

Isolation of the rat CDAP-1, CDAP-2, CDAP-3, CDAP-4, CDAP-5, and CDAP-6 cDNA

The invention is based, at least in part, on the discovery of five rat genes, referred to herein as CDAP-1, CDAP-2, CDAP-3, CDAP-4, CDAP-5, and CDAP-6. These genes were identified from rat cerebellar granule neurons undergoing programmed cell death, using a high-throughput differential display assay (RADE™). Over 240 primer pairs were utilized on cerebellar granule neuron RNA samples isolated at 0, 1, 3, 6, 12, and 24 hours after senimipotassium withdrawal for high-throughput differential-display analysis. Twenty-three differentially-expressed RADE bands were successfully gel-purified and subcloned. To verify and quantify differential expression of the RADE clones, programmed cell death templates were prepared for gridding onto high-density cDNA arrays. RADE clones encoding CDAP-1 and CDAP-6 were also verified by quantitative RT-PCR. For 16 RADE bands tested, 9 showed differential expression of more than 3-fold at 3 hours after initiation of cerebellar granule neuron programmed cell death. Sequence analysis indicated that two of the genes identified were previously known to be transcriptionally regulated by programmed cell death. One was the rat homolog of human protein phosphatase Wip1 previously shown to be induced by ionizing radiation in a p53-dependent fashion. The other has been shown to be specifically upregulated during cerebellar granule neuron programmed cell death and encodes for CPP-32, a cysteine protease central to the downstream caspase cascade of programmed cell death. The remaining 7 RADE bands fall into 6 sequence clusters indicative of 6 novel genes that are transcriptionally regulated by neuronal programmed cell death. The sequences of these clones were determined and termed CDAP-1, CDAP-2, CDAP-3, CDAP-4, CDAP-5, and CDAP-6.

The nucleotide sequence of the CDAP-1 nucleic acid molecule which is downregulated 2-fold at 3 hours after serum/potassium withdrawal, is shown in FIG. 1 and is set forth as SEQ ID NO: 1.

The nucleotide sequence of the CDAP-2 nucleic acid molecule which is upregulated 4-fold at 3 hours after serum/potassium withdrawal, is shown in FIG. 2 and is set forth as SEQ ID NO:2.

The nucleotide sequence of the CDAP-3 nucleic acid molecule which is upregulated 3-fold at 3 hours after serum/potassium withdrawal, is shown in FIG. 3 and is set forth as SEQ ID NO:3.

The nucleotide sequence of the CDAP-4 nucleic acid molecule which is upregulated 6-fold at 3 hours after serum/potassium withdrawal, is shown in FIG. 4 and is set forth as SEQ ID NO:4.

The nucleotide sequence of the CDAP-5 nucleic acid molecule which is upregulated 5-fold at 3 hours after serum/potassium withdrawal is shown in FIG. 5 and is set forth as SEQ ID NO:5. The partial length protein encoded by this nucleic acid comprises about 158 amino acids and has the amino acid sequence shown in FIG. 5 and set forth as SEQ ID NO:6.

The nucleotide sequence of the CDAP-6 nucleic acid molecule which is upregulated 4-16-fold at 3 hours after serum/potassium withdrawal, is shown in FIG. 6 and is set forth as SEQ ID NO:7.

Tissue Distribution of CDAP mRNA

This Example describes the tissue distribution of CDAP mRNA, as determined by Northern blot hybridization and in situ hybridization.

Northern blot hybridizations with the various RNA samples were performed under standard conditions and washed under stringent conditions, i.e., 0.2×SSC at 65° C. The DNA probe was radioactively labeled with ³²P-dCTP using the Prime-It kit (Stratagene, La Jolla, Calif.) according to the instructions of the supplier. Filters containing rat mRNA (Rat MultiTissue Northern Blot from Clontech, Palo Alto, Calif.) were probed in ExpressHyb hybridization solution (Clontech) and washed at high stringency according to manufacturer's recommendations.

For in situ analysis, various tissues obtained from adult Sprague-Dawley rats were first frozen on dry ice. Ten-micrometer-thick coronal sections of the tissues were postfixed with 4% formaldehyde in DEPC treated 1X phosphate-buffered saline at room temperature for 10 minutes before being rinsed twice in DEPC IX phosphate-buffered saline and once in 0.1 M triethanolamine-HCI (pH 8.0). Following incubation in 0.25% acetic anhydride-0.1 M triethanolamine-HCI for 10 minutes, sections were rinsed in DEPC 2X SSC (1X SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCi, 10 mM Tris (pH 7.5), 1 mM EDTA, 0.01% sheared salmon sperm DNA, 0.01% yeast tRNA, 0.05% yeast total RNA type XI, 1 X Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2 X SSC. Sections were then sequentially incubated at 37° C. in TNE (a solution containing 10 mM Tris-HCI (pH 7.6), 500 mM NaCl, and 1 mM EDTA), for 10 minutes, in TNE with 10 μg of RNase A per ml for 30 minutes, and finally in TNE for 10 minutes. Slides were then rinsed with 2 X SSC at room temperature, washed with 2 X SSC at 50° C. for 1 hour, washed with 0.2 X SSC at 55° C. for I hour, and 0.2 X SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

CDAP-2 and CDAP-3 message was detected in the brain. CDAP-4 message was detect in the brain (cerebellar granular cell layer), liver, kidney, and testis. CDAP-5 message was detect in the brain (cortex, hippocampus, cerebellar granular cell layer), eye, lung, brown fat, kidney, heart, spleen, liver, skeletal muscle, and kidney.

### Example 2

EXPRESSION OF RECOMBINANT CDAP PROTEIN IN BACTERIAL CELLS

In this example, CDAP is expressed as a recombinant glutathione-S-transferase (GST) fusion polypeptide in E. coli. and the fusion polypeptide is isolated and characterized. Specifically, CDAP is fused to GST and this fusion polypeptide is expressed in E. coli, e.g., strain PEB 199. Expression of the GST-CDAP fusion protein in PEB 199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 3

EXPRESSION OF RECOMBINANT CDAP PROTEIN IN COS CELLS

To express the CDAP gene in COS. cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire CDAP protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the CDAP DNA sequence is amplified by PCR using two primers. The 5′ primer contains the restriction site of interest followed by approximately twenty nucleotides of the CDAP coding sequence starting from the initiation codon; the 3′ end sequence contains complementary sequences to the other restriction site of interest, a translation stop codon, the HA tag or FLAG tag and the last 20 nucleotides of the CDAP coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the CDAP gene is inserted in the correct orientation. The ligation mixture is transformed into E. coli cells (strains HB 101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the CDAP-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the CDAP polypeptide is detected by radiolabelling (³⁵S-methionine or ³⁵S-cysteine available from NEN, Boston, MA, can be used) and imrnunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells arie labelled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the CDAP coding sequence is cloned directly into the polylinker of the pCDNA/Amp vector using the appropriate restriction sites. The resulting plasmid is transfected into COS cells in the manner described above, and the expression of the CDAP polypeptide is detected by radiolabelling and immunoprecipitation using a CDAP specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 60% homologous to the nucleotide sequence of SEQ ID NO: 1, 2, 3, 4, 5, or 7, or a complement thereof;
b) a nucleic acid molecule comprising a fragment of at least 420 nucleotides of a nucleic acid comprising the nucleotide sequence of SEQ ID NO: 1, 2, 3, 4, 5, or 7, or a complement thereof;
c) a nucleic acid molecule which encodes a polypeptide comprising an amino acid sequence at least about 60% homologous to the amino acid sequence of SEQ ID NO:6;
d) a nucleic acid molecule which encodes a fragment of a polypeptide comprising the amino acid sequence of SEQ ID NO:6, wherein the fragment comprises at least 15 contiguous amino acid residues of the amino acid sequence of SEQ ID NO:6; and
e) a nucleic acid molecule which encodes a naturally occurring allelic variant of a polypeptide comprising the amino acid sequence of SEQ ID NO:6, wherein the nucleic acid molecule hybridizes to a nucleic acid molecule comprising SEQ ID NO:5, under stringent conditions.

2. The isolated nucleic acid molecule of claim 1 which is selected from the group consisting of:
a) a nucleic acid molecule comprising the nucleotide sequence of SEQ ID NO:1, 2, 3, 4, 5, or 7, or a complement thereof; and
b) a nucleic acid molecule which encodes a polypeptide comprising the amino acid sequence of SEQ ID NO:6.

3. The nucleic acid molecule of claim 1, further comprising vector nucleic acid sequences.

4. The nucleic acid molecule of claim 1, further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:6, wherein the fragment comprises at least 15 contiguous amino acids of SEQ ID NO:6;
b) a naturally occurring allelic variant of a polypeptide comprising the amino acid sequence of SEQ ID NO:6, wherein the polypeptide is encoded by a nucleic acid molecule which hybridizes to a nucleic acid molecule comprising SEQ ID NO:5 under stringent conditions;
c) a polypeptide which is encoded by a nucleic acid molecule comprising a nucleotide sequence which is at least 60% homologous to a nucleic acid comprising the nucleotide sequence of SEQ ID NO: 1, 2, 3, 4, 5, or 7; and
d) a polypeptide comprising an amino acid sequence which is at least 60% homologous to the amino acid sequence of SEQ ID NO:6.

9. The isolated polypeptide of claim 8 comprising the amino acid sequence of SEQ ID NO:6.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:6;
b) a fragment of a polypeptide comprising the amino acid sequence of SEQ ID NO:6; and
c) a naturally occurring allelic variant of a polypeptide comprising the amino acid sequence of SEQ ID NO:6, wherein the polypeptide is encoded by a nucleic acid molecule which hybridizes to a nucleic acid molecule comprising SEQ ID NO:5 under stringent conditions;
comprising culturing the host cell of claim 5 under conditions in which the nucleic acid molecule is expressed.

13. A method for detecting the presence of a polypeptide of claim 8 in a sample comprising:
a) contacting the sample with a compound which selectively binds to the polypeptide; and
b) determining whether the compound binds to the polypeptide in the sample to thereby detect the presence of a polypeptide of claim 8 in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule in claim 1 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 1 in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising:
a) contacting the polypeptide, or a cell expressing the polypeptide with a test compound; and
b) determining whether the polypeptide binds to the test compound.

20. The method of claim 19, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for CDAP activity.

21. A method for modulating the activity of a polypeptide of claim 8 comprising contacting the polypeptide or a cell expressing the polypeptide with a compound which binds to the polypeptide in a sufficient concentration to modulate the activity of the polypeptide.

22. A method for identifying a compound which modulates the activity of a polypeptide of claim 8 comprising:
a) contacting a polypeptide of claim 8 with a test compound; and
b) determining the effect of the test compound on the activity of the polypeptide to thereby identify a compound which modulates the activity of the polypeptide.


SEQUENCE LISTING (7 sequences)

SEQ ID NO: 1 - DNA, 403 bp, Rattus norvegicus
cccttctgacgtcaccattgattgatgatacttgactgtgctctggatgtagttggtgctagttaaatacatatgttttcaaactccaactggatgttaagccgtgtctctgatattagtgtagggatgcttatggtgtaatataattttaagcagttctactattttaacatcaatgtacatctgcttgatctcaaattttggtcacagaaggcctggaaagaggtcttacgccacccagccattagctgtctgtcataccatgctcttcctgggggcagaaatgacatcaccaaagaaaatgtttgagtaaaaaagtgcccaagttccaaatgggtatttttagggggggtgactttaaaatgactatttttggtcatttatacccaaaaaaaaaaaaggg

SEQ ID NO: 2 - DNA, 664 bp, Rattus norvegicus
ggagaggtgccaacgaagaagcttccactctgtagcaggggcagaggaaaggacctcacacatgttcacgtgaattaggttgtgaatggtgctgcgtggtattgagtctgtaacctgtaaataacctggggaagtagtgttttagctcacccagaagcatctgtccctagttaacctgtagtataggacactgacgcactttccatgtaaaacctgggtttgggtcttcccaagggaccttggaagaatctcctcaggaggtctccctcctcacacgactccatgtaacagttccaagtaaactgagcggctttagttctggaaattccagttgaagctacagggctaacaccattaacacaagaagtgggttcacaaatttgcttttcctttaaaggtagtacccatgagcctaagctatagaatatagttggacttgcccttcattgttttcaaaaattccggggataatgtatatattggtgtcaagacctagttccctgactcatgtacacgtaggttttaataataggactttgttatgttattttttgttgttgtcgttaattacagtgtcttgggttcatcgtgtgaaggttctgctgggtaggatctcgcacctttaaaaaactgcctcttagttactctagtaagcccacaaatg

SEQ ID NO: 3 - DNA, 585 bp, Rattus norvegicus
ccgaacccagggccttgcgcttcctaggtaagcgctctaccgaacttttaattctgatgtcaccacctcccaagtgctgggataataggcatgtactgccatgccgagttttattcagggctctgtatcaaacccagggctttgggtaggctaggcaaacatgctaccaactgaactacatccagactcagccctagagccgatcaacatttcccacaaagttcctacaagtggctatcatgtcacacatgcagcaagggcatggctcctgtggttcgtcaccactcagcagcatgccacaaccttgtgtgtgaagcagggcttcagctcaggctgagcaagccaccctttgcctcctggtgctggcattatgggactgtgcaacatttccacagctctaccattctgacagcaatgcctaaaacttccaattatttccacatccttgcaaacacttgggtttttttgttttgttttgtttgtttgtttaagacagggtctctttgactagttctggctcgcctggaactcacagcattgttgggcttgaattcagagatctttctgacaccacacccggtgcta

SEQ ID NO: 4 - DNA, 1057 bp, Rattus norvegicus
agcggacaggaccagtgaagaagccacggtagctgctgccatctgctgccggagccggccttcggcaaaggcctcctgggttcaccagtgacagcctcaggcaggcattgtacctgtggctggacgcagagatggacgtcctggctctcttgtgtctagccaaaagtggggagactctgcctgggggaacttggcgtctcatcctgggtacccattcctggtgtatgtgtggggaagcacctccttcatggtcagggggcctgtgcttggccttctgccatcgaagatgttaagctatagttggctttggccagctgctccagtatatcagaacctgagagcacttgctacaaggctagtgttcaggccttaggcctccagagtgaatgtatcctgcaggaagataatgatggatcgtgacccttgacggtcacccccctcccccaggtcagatgtcaccagactagaacagtatctgaaagctgctggggccactcacagcttgcttactctggagacagcattttgggctccctgattaatgcagatcagttctgcccacctccaggggtggatccagctgtgaggctcacctgtatcttccagatgttctcatctgctgcaccgaaggctctggccctgctcaggagaacacgctacgaactcctagctgactctgtttgcactggagaaccacacagggcttaccccactaccctgtgcactgactggcttcactttatgaaggaagagacagggccagagaagcaatgtcatgcagccagtgatgctaggacataaatccagagtggctggccctgaagccatgcctcttggcaatgccaggctgggcatcctatttttgaagcaaacaaaaaatgagaggacaggctgtgcttcagcggcttgttcctggacctatgctcccttagccccagtcccacggattatgtggagagtggaggagcaacagagggcgactgtactaaggccacacaagtcgacaagaacacctatatccttttgacctcttctgcttttttatagtaagctttcc

SEQ ID NO: 5 - DNA, 692 bp, Rattus norvegicus
atcgtgataaagacagtgggagtagctcaccgttacccaagtatgcttcatctcccaaaccagacaacagctacatgttcaaacgggagcccccagagggatgtgagcgagtgaaggtctttgaggaaatggcgtctcgtcagcctatctcggcccctctcttttcatgtcctgacaaaaacaaggttaatttcatcccaaccggatcagctttctgtcctgtaaaacttctaggccctctcttacctgcctctgacctgatgctcaagaactctcctaattctggccagagctcagctctggccacactaaccgtagagcagctctcctcccgggtctccttcacgtccctttctgatgacaccagcaccgcagactccctggagccctctgtccagcagccatctcagcagcagcagctcctgcaggatttgcaggcagaggaacacatctccactcagaactatgtgatgatctaaagcagagggggagctggcctccgcccatgttccatggatcgggaatgagatctcagacatctatctgcatggagtgacaaactttctgaacaccaccaccaacagcaaaatacttagcatcataaaatagctattaacactgatcttggcagggaccgacttctattcagcagtttttgtggaaagcagtaatgcttgcaaaaatgtgcgtgt

SEQ ID NO: 6 - protein, 158 aa, Rattus norvegicus
RDKDSGSSSPLPKYASSPKPDNSYMFKREPPEGCERVKVFEEMASRQPISAPLFSCPDKNKVNFIPTGSAFCPVKLLGPLLPASDLMLKNSPNSCQSSALATLTVEQLSSRVSFTSLSDDTSTADSLEPSVQQPSQQQQLLQDLQAEEHISTQNYVMI

SEQ ID NO: 7 - DNA, 192 bp, Rattus norvegicus
cccttgagcaaggcagactggacaggaggcagtgagggttagtgggtgtgagctaaaatagactccagtagtccaggtggtaggagccaagaagcagagtcagaactgaccttgtagtttgagaaagtaaccacagcattcctgttgacaggtctgttcaccttagagtatccctccccgaaaaaaaaaaaa